(19) United States
(12) Patent Application Publication
(10) Pub. No.: US 2010/0242127 A1
(43) Pub. Date: Sep. 23, 2010

(54) METHODS TO IDENTIFY MODULATORS OF B-RAF PROTEIN KINASE AND THEIR USE FOR THE TREATMENT OF ANXIETY AND DEPRESSION

(75) Inventors: Christiane Hitz, Munchen (DE); Sabine Hölter, Munchen (DE); Ralf Kühn, Freising (DE); Wolfgang Wurst, Munchen (DE); Benedikt Wefers, Markt Schwaben (DE)

Correspondence Address: PILLSBURY WINTHROP SHAW PITTMAN LLP, ATTENTION: DOCKETING DEPARTMENT, P.O BOX 10500, McLean, VA, 22102, US

(73) Assignee: HELMHOLTZ ZENTRUM MUNCHEN, 85764 Neuherberg (DE)

(21) Appl. No.: 12/602,753

(22) PCT Filed: Jun. 3, 2008
(86) PCT No.: PCT/EP2008/004416

§ 371 Date: Jun. 4, 2010

Related U.S. Application Data
(60) Provisional application No. 60/941,846, filed on Jun. 4, 2007.

(51) Int. Cl. A01K 67/00; C12Q 1/68; C12Q 1/48; G01N 33/53; A61K 31/44; A61P 25/00; C07D 213/62

(52) U.S. Cl. 800/18; 435/6; 435/15; 435/7.1; 514/350; 546/298

(57) ABSTRACT

The present invention relates to a method for identifying a compound capable of modulating an anxiety or depression disorder comprising the steps of: (a) contacting a composition comprising a B-Raf protein or a B-Raf gene in expressible form or a transcript thereof with a compound under conditions that allow for an interaction of the B-Raf protein or the B-Raf gene or a transcript thereof and the compound; and (b) measuring whether said interaction, if any, results in (i) a change of B-Raf kinase activity compared to B-Raf kinase activity in the absence of said compound; (ii) a modulation of the expression of the B-Raf gene compared to B-Raf gene expression in the absence of said compound; or (iii) the formation of a complex between the compound and the B-Raf protein, wherein such a change in activity, modulation of expression or the formation of a complex is indicative of the compound being a modulator of an anxiety or depression disorder. Further, the invention relates to a method for treating an anxiety or depression disorder in an individual comprising administering to the individual an effective amount of a compound inhibiting B-Raf kinase activity or gene expression and to a use of a compound that inhibits B-Raf kinase activity or gene expression in the manufacture of a pharmaceutical composition for treating an anxiety or depression disorder. Moreover, the invention relates to a method of diagnosing a B-Raf associated anxiety or depression disorder and to a genetically engineered mouse. Finally, the invention also relates to a method of identifying another gene contributing to the pathophysiology of an anxiety or depression disorder apart from B-Raf.

The present invention relates to a method for identifying a compound capable of modulating an anxiety or depression disorder comprising the steps of: (a) contacting a composition comprising a B-Raf protein or a B-Raf gene or a transcript thereof in expressible form with a compound under conditions that allow for an interaction of the B-Raf protein or the B-Raf gene or a transcript thereof and the compound; and (b) measuring whether said interaction, if any, results in (i) a change of B-Raf kinase activity compared to B-Raf kinase activity in the absence of said compound; (ii) a modulation of the expression of the B-Raf gene compared to B-Raf gene expression in the absence of said compound; or (iii) the formation of a complex between the compound and the B-Raf protein, wherein such a change in activity, modulation of expression or the formation of a complex is indicative of the compound being a modulator of an anxiety or depression disorder. Further, the invention relates to a method for treating an anxiety or depression disorder in an individual comprising administering to the individual an effective amount of a compound inhibiting B-Raf kinase activity or gene expression and to a use of a compound that inhibits B-Raf kinase activity or gene expression in the manufacture of a pharmaceutical composition for treating an anxiety or depression disorder. Moreover, the invention relates to a method of diagnosing a B-Raf associated anxiety or depression disorder and to a genetically engineered mouse. Finally, the invention also relates to a method of identifying another gene contributing to the pathophysiology of an anxiety or depression disorder apart from B-Raf.

Several documents are cited throughout this specification. The complete disclosure content of the documents cited herein (including manufacturer's specifications, instructions, etc.) is herewith incorporated by reference.

Depression and anxiety disorders represent some of the most common and proliferating health problems worldwide (Wong and Licinio, Nat Rev Neurosci, 2, 343-351 (2001)). Both types are serious medical illnesses that affect about 14% of the European population at some point in their lifetime (Alonso, et al., Acta Psychiatr Scand Suppl, 21-27 (2004)) and unipolar depression is predicted to become the second most prevalent cause of illness-induced disability by 2020 (Mathers and Loncar, PLoS Med, 3, e442 (2006)) (Lopez and Murray, Nat Med, 4, 1241-1243 (1998)). Anxiety disorders and depression have been regarded as separate clinical entities, predominantly because different drug treatments have been used to treat the diseases, usually tricyclic antidepressants that target noradrenaline and/or serotonin transporters and benzodiazepines that act via GABA-A receptors, respectively (Shorter and Tyrer, Bmj, 327, 158-160 (2003)). However, clinically the two disorders exhibit a considerable comorbidity (Merikangas, et al., Arch Gen Psychiatry, 60, 993-1000 (2003)) and a continuum model from anxiety syndromes to mild, moderate, and severe depression was proposed (Wong and Licinio, Nat Rev Neurosci, 2, 343-351 (2001)). Drugs that are effective in both conditions would be particularly beneficial and cost effective.

Natural anxiety is accompanied by a characteristic set of behavioural and physiological responses including avoidance, vigilance, and arousal, which evolved to protect the individual from danger. These anxiety-related responses are known in higher animals and are part of a universal mechanism by which organisms adapt to adverse conditions. In its pathological form, anxiety can severely interfere with normal life, and has been classified into six disorders described in the Diagnostic and Statistical Manual of the American Psychiatric Association: generalised anxiety disorder, social phobia, simple phobia, panic disorder, post-traumatic stress disorder (PTSD), and obsessive-compulsive disorder (OCD) (American Psychiatric Association, Diagnostic and statistical manual of mental disorders, 4^{th} ed. 1994, American Psychiatric Press, Washington D.C.). Despite the wide range encompassed by these six disorders, all of them probably share common behavioural and physiological characteristics since most anxiety disorders respond to a similar spectrum of pharmacological treatments (Bourin and Hascoet, Curr Opin Investig Drugs, 2, 259-265 (2001)).

Abnormal emotion is also frequently seen in other neuropsychiatric and neurological diagnoses and can frequently precipitate symptoms in these conditions. Current treatments for depression and anxiety disorders are of limited efficacy in a considerable proportion of patients, and are associated with troublesome side-effects that reduce compliance in many other patients (Wong and Licinio, Nat Rev Drug Discov, 3, 136-151 (2004)) (Holmes, et al., Trends Pharmacol Sci, 24, 580-588 (2003)). A better understanding of the pathophysiology of these disorders and the development of novel, improved therapeutic treatments would fill a considerable unmet medical need.

A key factor in the lack of rational therapeutic intervention for anxiety and depression disorders is the limited knowledge of the etiology and pathophysiology that underlie these conditions. Despite this lack of knowledge, depression responds to a range of antidepressant medications. Almost all available medications for depression are based on discoveries made more than five decades ago and are based on tricyclic antidepressants which act by inhibiting the plasma membrane transporters for serotonin and/or noradrenaline. These compounds provided a template for the development of new classes of antidepressants, including the SSRIs (selective serotonin reuptake inhibitors), NRIs (noradrenaline reuptake inhibitors), and SNRIs (serotonin and noradrenaline reuptake inhibitors). Since these compounds have the same mechanism of action as the older tricyclics, their efficacy and successful therapeutic range remains the same. These medications require a period of several weeks before their action becomes manifest. Despite intense research, the changes that these drugs induce in the brain and that underlie their therapeutic action remain obscure. Due to the long period to achieve clinical benefit, side-effects and a response in less than half of patients showing full remission, the current medications for depression are not ideal (Berton and Nestler, Nat Rev Neurosci, 7, 137-151 (2006)). The most common and successful therapy over the last four decades for the majority of patients suffering from anxiety disorders is treatment with benzodiazepines. Benzodiazepines have come under attack over recent years because of their abuse liability, withdrawal reactions, and development of tolerance. The problems associated with their use prompted the research for alternative agents. Old classes of antidepressants, such as tricyclic antidepressants and new classes like SSRIs appear useful in some anxiety states, and their favourable side-effect profile has elevated their use in these conditions. However, the ideal anxiolytic has not been developed (Argyropoulos, et al., Pharmacol Ther, 88, 213-227 (2000)). Thus, for both anxiety and depression disorders a need for safer and more effective treatment exists. Due to the lack of information on the etiology and pathophysiology of anxiety and depression diseases, the art has not developed any means to rationally optimise the pharmacotherapy of these conditions.

Therefore, the technical problem underlying the present invention was to identify more appropriate or further means that allow for the development of drugs useful in the treatment of anxiety and/or depression.

The solution to this technical problem is achieved by providing the embodiments characterized in the claims.

Accordingly, the present invention relates to a method for identifying a compound capable of modulating an anxiety or depression disorder comprising the steps of:
(a) contacting a composition comprising a B-Raf protein or a B-Raf gene in expressible form or a transcript thereof with a compound under conditions that allow for an interaction of the B-Rat protein or the B-Raf gene or a transcript thereof and the compound; and
(b) measuring whether said interaction, if any, results in
   i. a change of B-Raf kinase activity compared to B-Raf kinase activity in the absence of said compound;
   ii. a modulation of the expression of the B-Raf gene compared to B-Raf gene expression in the absence of said compound; or
   iii. the formation of a complex between the compound and the B-Raf protein,
      wherein such a change in activity, modulation of expression or the formation of a complex is indicative of the compound being a modulator of an anxiety or depression disorder.

The term “compound” to be employed in the method of the invention includes a single substance or a plurality of substances. Said compound(s), inter alia, may be chemically synthesized, recombinantly produced or produced via microbial fermentation. It can also be comprised in, for example, samples, e.g., cell extracts from, e.g., plants, animals or microorganisms. Moreover, the compound to be screened can be contained in libraries of small molecules, such as organic or inorganic small molecules. Suitable libraries for small molecules are commercially available, for example from ChemBridge Corp., San Diego, USA. In addition, libraries comprising antibodies or functional fragments or derivatives thereof (i.e. fragments or derivatives maintaining the binding specificity of the original antibody) may be used as a starting point in the screening process. Also, libraries of aptamers such as peptide aptamers might be employed. The skilled artisan is of course free to use any other starting point of desired compounds for use in the screening assays described throughout the specification.

If a sample containing (a) compound(s) is identified in the method of the invention, then it is either possible to isolate the compound from the original sample identified as containing the compound in question or one can further subdivide the original sample, for example, if it consists of a plurality of different compounds, so as to reduce the number of different substances per sample and repeat the method with the subdivisions of the original sample. It can then be determined whether said sample or compound displays the desired properties, for example, by the methods described herein. Depending on the complexity of the samples, the steps described above can be performed several times, preferably until the sample identified according to the method of the invention only comprises a limited number of or only one substance(s). Preferably said sample comprises substances of similar chemical and/or physical properties. Once the person skilled in the art has become acquainted with the method of the present invention, he can without further ado perform this method and design modifications thereof, for example in accordance with other cell based assays described in the prior art. Furthermore, the person skilled in the art will readily recognize which further classes of compounds may be used in order to perform the method of the invention. For example, enzymes that convert a certain precursor into a compound may be employed wherein the compound is then used in the method of the invention. Such adaptations of the method of the invention are well within the skill of the person skilled in the art and can be performed without undue experimentation.

The term “modulation of an anxiety or depression disorder” is used according to the present invention to describe a measurable change resulting either in an increase or a decrease of the severity of symptoms, or the presence of additional symptoms or a lack of specific symptoms or a total lack of symptoms. In other words, any change of the symptoms which is causally related to the interaction with the compound when compared to symptoms in the absence of said compound is encompassed by the above term. Generally, it is preferred that the modulation is an alleviation or elimination.

The term “a composition comprising a B-Raf protein or a B-Raf gene in expressible form or a transcript thereof” as used in the context of the invention describes a composition that can be of or comprises any material/substance or plurality of materials/substances which does not alter or interfere with the natural molecular conformation and/or activity of B-Raf protein and allows for interaction with the compound to be screened for under appropriate conditions. It also refers to any composition that allows for the expression of the B-Raf gene. Preferably, said composition is of liquid nature. The composition may thus comprise the above recited compound and the B-Raf protein which are contained in a solution preferably reflecting physiological conditions. Said solution comprising said compound is preferably an aqueous solution. More preferred, said aqueous solution is buffered. Buffers are well known in the art and the skilled person is aware of appropriate buffers in dependency of the substances being assayed. Furthermore, ionic strength may be adjusted, e.g., by the addition of sodium chloride. The concentration of sodium chloride is between 0 and 2 M, preferably between 100 and 200 mM. Alternatively, sodium chloride is absent from the assay. For biological assays in many cases the presence of further substances, including other salts than sodium chloride, trace elements, amino acids, vitamins, growth factors, ubiquitous co-factors such as ATP or GTP, is required. Said further substances may either be added individually or provided in complex mixtures such as serum. These and further accessory substances are well known in the art as are concentrations suitable for biological assays. Minimally the composition comprises either B-Raf protein, the B-Raf gene in expressible form or a transcript thereof, optionally in combination with the means allowing for expression of functional B-Raf protein. For example, such a composition comprises a B-Raf protein in an aqueous solution, preferably a physiological solution. Alternatively, the composition may comprise the B-Raf gene in expressible form or a transcript thereof in combination with the means allowing for expression of functional B-Raf protein. Such means are for example, a suitable cell or tissue. The above material can further for example be (sepharose) beads, a membrane, a glass-, polypropylene- or silicon chip. A B-Raf gene in expressible form is according to the invention is a sequence containing any features that allow for expression of functional B-Raf protein in any expression system. Said sequence may be part of a vector and said vector containing the sequence may be stably or transiently transfected in a prokaryotic or eukaryotic cell in order to produce functional B-Raf protein.

The term “B-Raf gene” and “B-Raf protein” refers to the structure and coding sequence of the B-Raf gene and its isoforms as well as its gene product all of which have been reported (Sithanandam, et al., Oncogene, 5, 1775-1780 (1990); Ikawa, et al., Mol Cell Biol, 8, 2651-2654 (1988); Papin, et al., J Biol Chem, 273, 24939-24947 (1998); Barnier, et al., J Biol Chem, 270, 23381-23389 (1995)). The mouse and human B-Raf sequences are reported in GenBank with the accession numbers NM_{—}139294 and NM_{—}004333, respectively, and the coding and protein sequences are depicted in SEQ ID NO: 1, SEQ ID NO: 2, SEQ ID NO: 3 and SEQ ID NO: 4 respectively.

The term “B-Raf gene in expressible form” includes the above-described B-Rat gene itself including parts thereof essential to achieve expression of a functional B-Raf protein such as the promoter, the start and stop codon. Alternatively, the term also refers to sequences artificially linked to the open reading frame of the B-Raf gene which allow for expression, such as for example in the context of a vector a promoter or enhancer sequences or any other sequences that lead in the context of the prokaryotic or eukaryotic protein expression apparatus to the expression of functional protein.

The term “conditions that allow for an interaction of B-Raf protein or the B-Raf gene in expressible form or a transcript thereof with a compound” describes in the context of the invention any condition that does allow the interaction of the above recited elements with said compound. For example, these conditions do not alter or interfere with the natural molecular conformation and/or activity of B-Raf protein such as physiological conditions. Advantageously, said conditions are conditions that maintain cell or tissue viability when applied. Cell viability, if necessary, may also be maintained by additional means, for example, addition of buffering media or agents. Additionally, said conditions allow e.g. a binding, optionally an inhibition of the compound with the gene or a part thereof, a transcript thereof or the translation (product) thereof. Interaction may be direct or mediated by one or a plurality of endogenous or added mediators.

The term “protein” describes an amino acid chain of more than 30 consecutive amino acids. The term “protein” is interchangeably used in connection with this invention with the term “polypeptide”. Both terms confer the same meaning. Moreover, what is comprised by said terms is in accordance with standard textbook knowledge.

The identification of a compound that modulates an anxiety or depression disorder may involve measuring B-Raf-mediated protein kinase activity, B-Raf expression, B-Raf mediated processes or complex formation as referred to hereinabove. Measuring the B-Raf mediated kinase activity can be done in vivo, ex vivo or in vitro. B-Raf mediated kinase activity can be measured, for example, by determining the level (used herein to refer to either amount or rate) of phosphorylation of Mek1 or Mek2 protein (Wellbrock, et al., Nat Rev Mol Cell Biol, 5, 875-885 (2004)). In general, kinase activity can be measured by providing a substrate which can be phosphorylated and determining the rate of phosphorylation events by for example change of colour of the substrate or level of radioactivity. These assays are well known to the skilled person and include, e.g., ELISA-based kinase activity assays, K-LISA™, Omnia™ Kinase Assay (Invitrogen), or antibody based kinase activity assays.

Complex formation of two substances can be examined with several methods. One is visual examination with or without visual aids, such as a microscope. Others include determining an increase in molecular weight of one of the substances, determining in supernatant the amount of a substance which has been added to a second immobilized substance and comparing it to the amount initially added, detecting a colour change upon complex formation, using ELISA methods, inter alia.

Before investing into clinical trials, pharmaceutical companies seek validation that a biological target is relevant to the disease and that a new compound designed to alter its function will perform in a safe manner in vivo. Of central importance for this approach is the availability of valid behavioural test procedures in animals for evaluating the potential efficacy of novel pharmacotherapeutics.

Various mouse mutants have been reported to exhibit phenotypes of abnormal depression or anxiety-related behaviour (Finn, et al., Neurogenetics, 4, 109-135 (2003)) (Cryan, et al., Trends Pharmacol Sci, 23, 238-245 (2002)). In some cases these phenotypes were predictable from existing knowledge like the phenotype found in noradrenaline transporter knockout mice that fits the profile of antidepressant efficacy of drugs that antagonise its function (Xu, et al., Nat Neurosci, 3, 465-471 (2000)). In other examples of mice with the knockout of specific receptors, like mGluR7 and GAL-R1, the mutants revealed novel mechanisms that subserve emotion and that highlight these gene products as potential novel therapeutic targets (Holmes, et al., Neuropsychopharmacology, 28, 1031-1044 (2003)) (Cryan, et al., Eur J Neurosci, 17, 2409-2417 (2003)). Such findings in mutant mice are particularly valuable when pharmacological agonists and antagonists are not available or impractical to study the function of a specific gene product. A good example for this are mice with engineered mutations in the GABA-A receptor leading to the development of novel anxiolytics that target specific subunits of the receptor with reduced sedative side-effects (Rudolph and Mohler, Annu Rev Pharmacol Toxicol, 44, 475-498 (2004)).

Although a mouse is not just a smaller version of a human, the brain of all vertebrates shows a common structural organisation. Among the mammalian brain the neural structures and the interconnecting circuits have marked similarities and most fundamental physiological and behavioural responses are evolutionary conserved. Of central importance for using mice to understand human behaviour and diseases is the validity of experimental procedures used to assess anxiety and depression-related behaviour. Specific criteria to evaluate such procedures are: (1) a reasonable analogy of the test behaviour to the human disorder in its manifestation or symptomotology has to be given; (2) a behavioural change that can be monitored objectively has to be subject to the test paradigm; (3) the test procedure must report behavioural changes that are reversed by the same treatment shown to be effective in humans; and (4) the test has to be reproducible between investigators (McKinney and Bunney, Arch Gen Psychiatry, 21, 240-248 (1969)).

Based on these principles it is possible to study anxiety- and depression-related phenotypes in the mouse using specific behavioural test paradigms (Cryan and Holmes, Nat Rev Drug Discov, 4, 775-790 (2005)). To measure anxiety responses the innate aversion of mice to exposed, well-lit spaces can be used. The aversive areas are represented differently in different tests like open, elevated arms in the elevated plus-maze or a light compartment in the light/dark exploration test (Belzung and Griebel, Behav Brain Res, 125, 141-149 (2001)) (Bourin and Hascoet, Eur J Pharmacol, 463, 55-65 (2003)). Over a test session wild-type mice are expected to avoid these aversive areas and to prefer to remain in the protected zones of the test device for most of the observation time. Both of these anxiety tests have shown predictive validity such that avoidance behaviour is reduced by treatment with clinically effective anxiolytics, mainly by benzodiazepines (Bourin and Hascoet, Curr Opin lnvestig Drugs, 2, 259-265 (2001)) (Rodgers and Dalvi, Neurosci Biobehav Rev, 21, 801-810 (1997)) (Rodgers, Behav Pharmacol, 8, 477-496 (1997)). With this rationale, phenotypic alterations, found with these tests in mutant mice in relation to wild-type controls, are interpreted as a reduced anxiety-like behaviour or an anxiolytic-like phenotype.

Likewise, the commonly used test procedures to assess depression-related behaviour in mice, the forced swim test (FST) and the tail suspension test (TST), are validated by the finding that administration of clinically effective antidepressants causes mice to actively and persistently engage in escape-directed behaviour for a longer time as compared to vehicle treated control animals (Cryan, et al., Trends Pharmacol Sci, 23, 238-245 (2002)) (Cryan, et al., Neurosci Biobehav Rev, 29, 571-625 (2005)). The FST is based on the observation that mice, placed in an inescapable cylinder filled with water, initially engage in escape-oriented movements, but exhibit increasing signs of immobility within minutes. The TST is a related task for behavioural despair, in which mice hang upside down by their tail and exhibit passive immobility after minutes of intense struggling. On this basis, these tests are used as phenotypic screens for depression-related behaviours of mutant mice, with decreases in basal immobility interpreted as an antidepressant-like phenotype. Thus, compounds identified in the method of the invention that modify an anxiety or depression disorder may be subsequently employed in these test systems for further validation.

One of the areas of further interest in accordance with the present invention is the Ras-Raf-Mek-Erk/MAPK pathway, which is an evolutionarily conserved protein kinase signal transduction pathway that is involved in the control of many fundamental cellular processes that include cell proliferation, survival, differentiation, apoptosis, motility, and metabolism (Garrington and Johnson, Curr Opin Cell Biol, 11, 211-218 (1999)) (Seger and Krebs, Faseb J, 9, 726-735 (1995)). The Erk/MAPK pathway mediates the transduction of extracellular signals from cell surface receptors to Erk/MAPK, which distributes them to different effectors. Many cell surface receptors induce the activation of Ras, a small GTPase that binds to and recruits Raf kinases to the cell membrane for subsequent activation. Activated Raf kinases are the point of entry into a three-layered kinase cascade in which Raf phosphorylates and activates Mek kinases (MAPK/Erk kinases), and Mek phosphorylates and activates Erk/MAPK. The substrates of Erk/MAPK are very diverse and include both cytosolic and nuclear localised proteins. A central function of the MAPK pathway is the activation of gene expression, mediated via phosphorylation of transcription factors. In different cell types MAPK signalling can be interpreted differently in a cell type-specific context, e.g., in PC12 cells sustained MAPK activation leads to terminal differentiation, while in fibroblasts it is required for mitogenesis. An additional level of complexity has been added by the finding that a number of scaffolding proteins and endogenous inhibitors interact with components of the MAPK pathway and their roles in regulating MAPK signalling are just emerging (Kolch, Nat Rev Mol Cell Biol, 6, 827-837 (2005)). Furthermore, each component may also fulfil functions outside the canonical MAPK pathway via crosstalking to other signalling molecules. Recent results imply that C-Raf acts in a kinase independent manner to control apoptosis and cell migration (Hindley and Kolch, J Cell Sci, 115, 1575-1581 (2002)). For B-Raf it has been shown that kinase impaired mutants identified in tumour cells exhibit a similar oncogenic potential as mutants that show several hundred fold increased kinase activity (Garnett and Marais, Cancer Cell, 6, 313-319 (2004)).

Another hallmark of the MAPK pathway is the presence of multiple isoforms at each level, which exhibit also tissue specific expression patterns. Eight different Erk isoforms were described, among which Erk1, Erk2, Erk3, Erk4, Erk5 and Erk7 are expressed in the adult rodent brain. Among these, Erk1, Erk2, Erk3 and Erk5 belong to the canonical MAPK signalling pathway. Regarding the Mek proteins, which activate Erks, Mek1 and Mek2 were identified as specific activators of Erk1 and Erk2. As activators of Meks, three Raf kinases, A-Raf, B-Raf and C-Raf were identified in mammalian cells (Wellbrock, et al., Nat Rev Mol Cell Biol, 5, 875-885 (2004)). Among them only B-Raf and C-Raf are expressed in the adult rodent brain (Storm, et al., Oncogene, 5, 345-351 (1990)). While C-Raf is ubiquitously expressed also in peripheral tissues, the expression of B-Raf in adults is mostly restricted to the brain and spinal chord, where multiple, alternatively spliced B-Raf isoforms have been reported (Storm, et al., Oncogene, 5, 345-351 (1990)) (Papin, et al., J Biol Chem, 273, 24939-24947 (1998)) (Barnier, et al., J Biol Chem, 270, 23381-23389 (1995)). The expression of B-Raf in the adult mouse brain is strongest in neurons of the cortex, the hippocampal CA1-3 regions, and the amygdalar nuclei (Di Benedetto, et al., J Comp Neurol, 500, 542-556 (2007)).

The functional role of B-Raf in the adult mouse brain has long been occluded since the complete knockout of the B-Raf gene leads to embryonic lethality (Wojnowski, et al., Nat Genet, 16, 293-297 (1997)) and chemical B-Raf inhibitors were not available. With the recent development of a conditional B-Raf mouse mutant it was possible to inactivate the B-Raf gene postnatally in neurons of the forebrain (Chen, et al., J Neurosci Res, 83, 28-38 (2006)). These mutants were studied in learning and memory tasks and revealed an essential role for B-Raf in the activation of Erk1 and Erk2, hippocampal synaptic plasticity, and hippocampus-dependent learning and memory. The conditional inactivation of B-Raf in the prenatal brain leads to a severe growth retardation due to impaired hypothalamic function and to early death (Zhong, et al., Nat Neurosci, (2007)).

Knockout mice for the Erk1 or Mek2 gene are viable and do not show strong phenotypes, possibly as result of a functional redundancy of Erk1 with Erk2 and of Mek2 with Mek1 (Seicher, et al., Learn Mem, 8, 11-19 (2001)) (Belanger, et al., Mol Cell Biol, 23, 4778-4787 (2003)). Since complete knockout mice for Mek1 and Erk2 exhibit embryonic lethality, the role of these proteins in the adult brain has not yet been studied with genetic models (Giroux, et al., Curr Biol, 9, 369-372 (1999)) (Yao, et al., Proc Natl Acad Sci USA, 100, 12759-12764 (2003)). Conditional mouse mutants have recently been described for both genes but have not yet been used to inactivate Mek1 or Erk2 specifically in the brain (Fischer, et al., Immunity, 23, 431-443 (2005)) (Galabova-Kovacs, et al., Cell Cycle, 5, 1514-1518 (2006)). Therefore, the knowledge about the role of individual components of the MAPK signalling pathway in the adult brain is presently limited and most evidence is based on results from neuronal cell or organotypic slice cultures and the use of chemical Mek inhibitors (Thomas and Huganir, Nat Rev Neurosci, 5, 173-183 (2004)). In transgenic mice expressing a dominant negative form of Mek1 in the postnatal forebrain, deficits in synaptic plasticity and memory retention were found (Kelleher, et al., Cell, 116, 467-479 (2004)). The acute blockade of Mek with the inhibitor PD184161 in the mouse brain was found to produce a depressive-like phenotype and to counteract the behavioural actions of antidepressants (Duman, et al., Biol Psychiatry, 61, 661-670 (2007)). Einat (Einat, et al., J Neurosci, 23, 7311-7316 (2003)) described that the mood stabilisers lithium and valproate stimulate Erk activation in the rat brain, while the inhibition of Mek with SL327 produced a manic-like effect; it has been proposed that Erk activation may mediate the anti-manic effects of mood stabilisers.

Besides studying the function of 8-Raf in neurons, a growing body of literature is focused on B-Raf as a human oncogene (Gamett and Marais, Cancer Cell, 6, 313-319 (2004)) (Dhomen and Marais, Curr Opin Genet Dev, 17, 31-39 (2007)) (Zebisch and Troppmair, Cell Mol Life Sci, 63, 1314-1330 (2006)). The highest incidence of oncogenic B-Raf mutations is found in melanoma, thyroid, colorectal, and ovarian cancer. The predominating mutation (V599E) destabilises the inactive B-Raf conformation and exhibits >500-fold increased in vitro kinase activity (Gamett and Marais, Cancer Cell, 6, 313-319 (2004)). Due to these findings, efforts have been taken to develop anticancer strategies that target Raf dependent signalling pathways. Several classes of small molecules are currently being optimised; most of the compounds directed at Raf also inhibit a range of other kinases. Of the Raf inhibitors in development, sorafenib (Nexavar) is most advanced and is used for the treatment of renal cell carcinoma (Schreck and Rapp, Int J Cancer, 119, 2261-2271 (2006)).

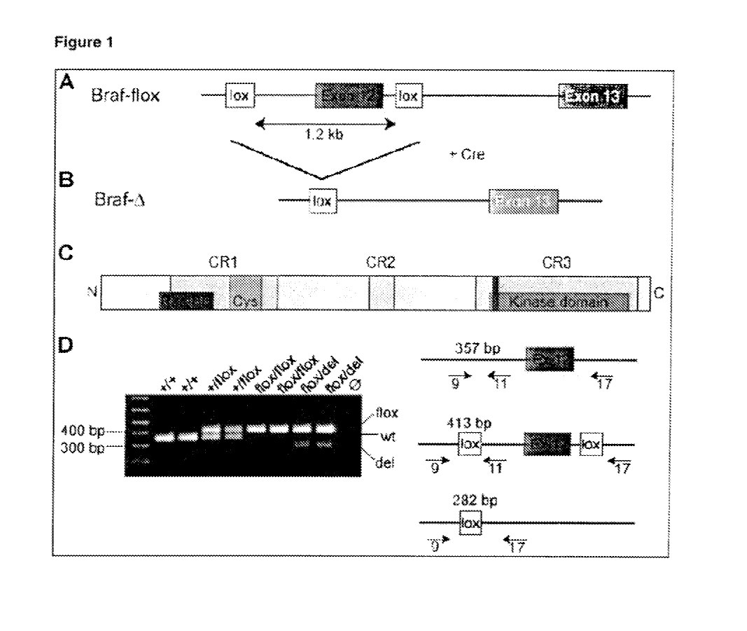

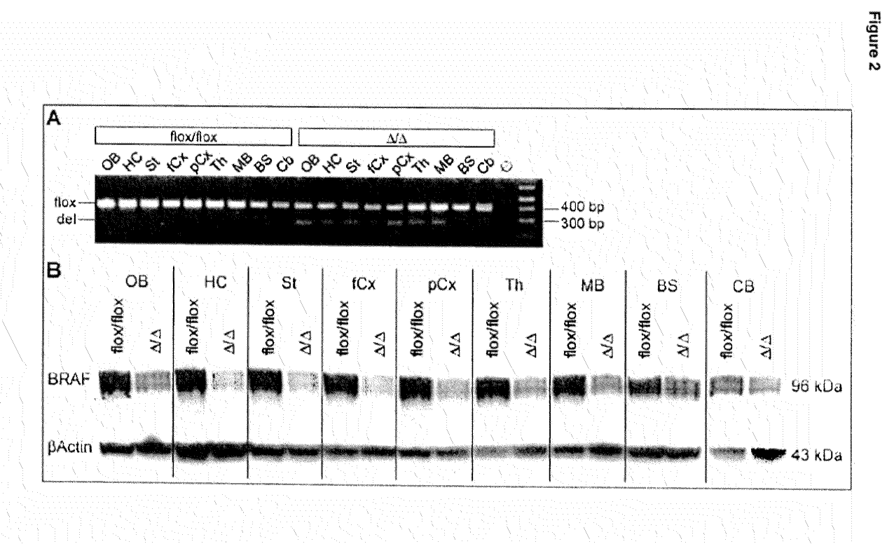

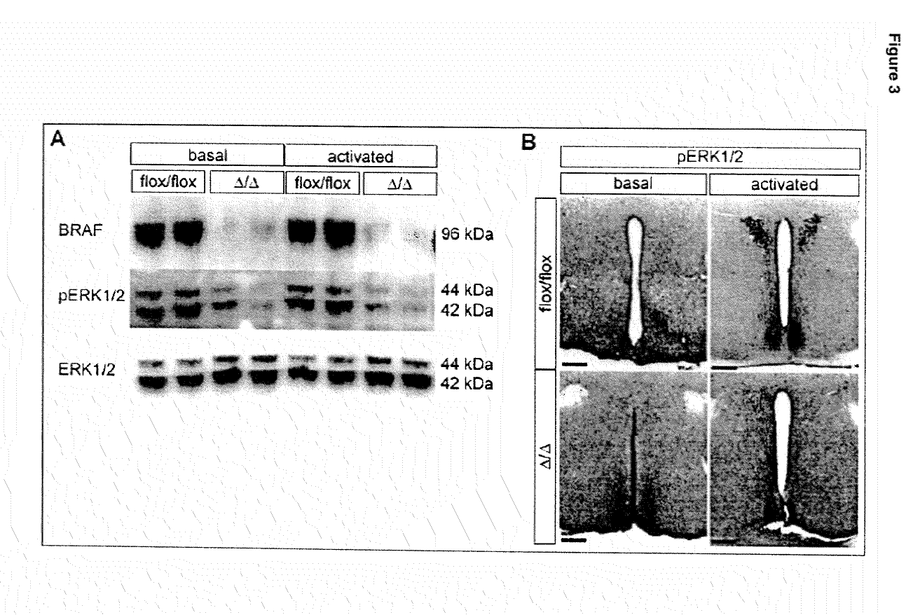

In accordance with the present invention it was surprisingly found that B-Raf is involved in the etiology and pathophysiology of anxiety and depression disorders. In an effort to study the involvement of B-Raf in said disorders a B-Raf conditional knockout mice was generated, wherein exon 12, which is the first exon encoding the kinase domain of B-Raf, is flanked by loxP sites to be deleted upon Cre mediated recombination (FIG. 1). The generation of this conditional knockout mouse was achieved according to the method described hereinafter, viz. crossing the transgenic mouse line B-raf-flox in which exon 12 of the B-Raf gene is flanked by two loxP sequences (cf. FIG. 1) described and manufactured by Chen, et al. (J Neurosci Res, 83, 28-38 (2006)) to the transgenic mouse line CamKII-CRE-159 that expresses Cre recombinase under the control of the CamKIIα promoter described and manufactured by Minichiello, et al. (Neuron, 24, 401-414 (1999)). Since the deletion inserts a reading frame shift in the coding region and a premature stop codon, the resulting protein is truncated and harbours no kinase domain any more. Upon crossing the Braf-flox mice to mice expressing Cre recombinase from the CamKIIa promoter, deletion of exon 12 occurred specifically in the forebrain of double transgenic CamKII-cre/Braf^{flox/flox} offspring (FIG. 2). This modification results in a loss of activation of downstream molecules of the MAPK cascade in the corresponding regions, as shown in FIG. 3.

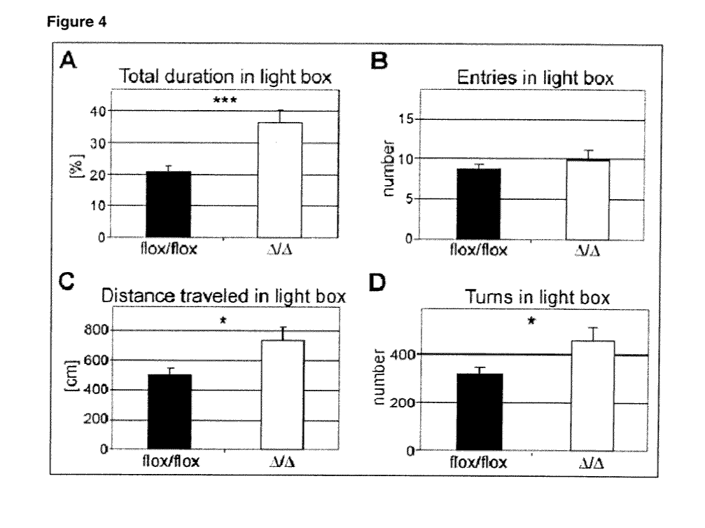

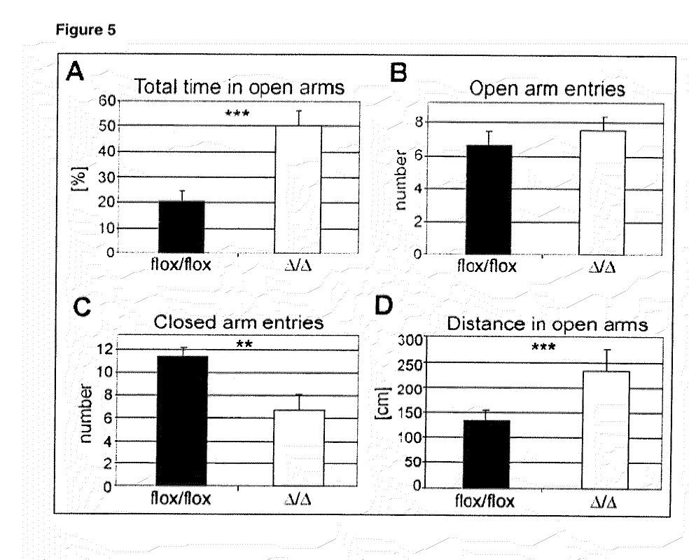

Anxiety related behaviour of CamKII-cre/Braf^{flox/flox} mutants and Braf^{flox/flox} controls was first analyzed with the Light-Dark-exploration test (n=11-15 mice for each group). In this task, highly significant genotype specific effects were found for CamKII-cre/Braf^{flox/flox} mice. As shown in FIG. 4, mutant mice of both sexes spent significantly more time in the light compartment of the box than control animals (ANOVA: p<0.001). However, the number of entries to the light compartment was not altered, indicating an increased duration of each visit of the light box. These observations showed that mutant B-Raf^{flox/flox}/CamKII-cre mice of both sexes had an increased preference for the aversive light compartment than controls. This finding is supported by an increased activity in the light box. Mutant mice traveled a significantly longer distance in the light box (ANOVA: p<0.05) and turned more often in this aversive compartment (ANOVA: p<0.05). Also in a second task for the assessment of anxiety related behaviour, the elevated plus maze (n=8-16 mice for each group), strong genotype specific effects were found for B-Raf^{flox/flox}/CamKII-cre mice. As shown in FIG. 5, mutant mice of both sexes spent significantly more time in the open arms of the maze than control animals (ANOVA: p<0.001). However, the number of entries to the open arms was not altered, indicating an increased duration of each open arm visit. Only the number of entries to the closed arms was decreased in mutants (ANOVA: p<0.01). These observations showed that mutant B-Raf^{flox/flox}/CamKII-cre mice of both sexes had an increased preference for the aversive open arms than their control littermates. This fact is supported by an enlarged distance mutants traveled in the open arms (ANOVA: p<0.001). All these results indicate a strongly reduced anxiety related behaviour in mice lacking B-Raf in forebrain neurons.

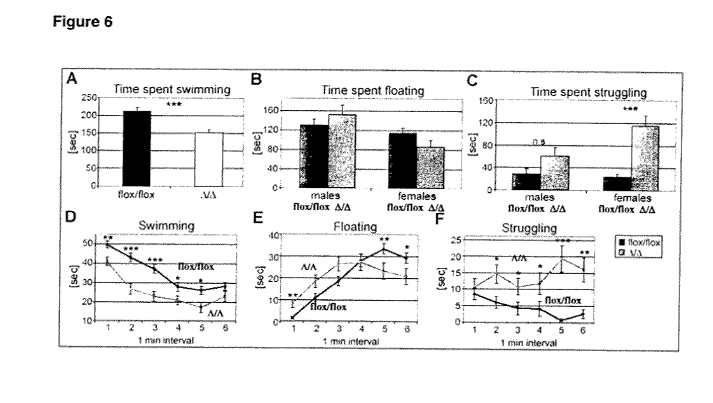

As shown in FIG. 6, the forced swim test (n=15-16 mice for each group) for the assessment of motivation and behavioural despair revealed significant genotype effects. Mutants of both sexes spent less time actively swimming (ANOVA: p<0.001). For the total time spent floating (passive behaviour) no genotype effect could be detected. However, looking at different time points during the test phase, a genotype effect was observed for the second half of the test phase. Mutant mice spent less time floating in the last two minutes of the test (ANOVA: p<0.01). For the time spent struggling (active escaping behaviour) throughout the entire test phase, a genotype effect manifested only in females, since female mutants spent significantly more time struggling than controls (ANOVA: p<0.001), whereas male mutants struggled only tendentially longer. The enlarged struggling of mutant mice was even more prominent in the second half of the test phase. Whereas control animals gave up in the last two minutes, mutants even increased their struggling effort at the same time (ANOVA: p<0.001). All these results show a decreased behavioural despair in B-Raf deficient mice, indicating antidepressive behaviour.

Taken together, the behavioural analyses surprisingly revealed antidepressive and a strongly reduced anxiety related behaviour in mice lacking B-Raf in forebrain neurons.

The novel findings described herein demonstrate that inhibition of B-Raf activity or expression leads to a reduction of anxiety and depression behaviour, and therefore agents that inhibit B-Raf activity or expression are useful in reducing the manifestation of pathological anxiety and depression behaviour. In an attempt to elucidate the potential mechanistic implications of the MAPK/ERK pathway in the behavioral changes of B-Raf lacking mice, it is contemplated that the signal cascade is interrupted and hence, whereas the applicant does not wish to be bound or limited by any specific theory, the anxiolytic phenotype is considered to be the consequence of a direct or indirect correlation between the GABA-A receptor and the MAP/ERK pathway (cf. Example 3). This finding provides a novel cause-and-effect relationship on a molecular level for an anxiety or depression disorder and is a major step in the direction of developing novel, safe anxiolytic and anti-depressive drugs without the common side-effects and therapeutic disadvantages of the presently used drugs and also provides new therapeutic strategies and diagnostic possibilities.

In a preferred embodiment of the above method of the invention, said composition, contains a viable cell comprising said B-Raf protein or said B-Raf gene in an expressible form.

Viable cells are preferred over, e.g. in vitro translation systems, due to the fact that viable cells more properly reflect an in vivo situation such as an in vivo situation in animals or humans. Viable cells are also preferred because the activity of the compound can easily be measured on three different levels: at the level of transcription, at the level of translation as well as at the level of protein activity. In one embodiment and if measuring is to be carried out at the transcription level, it is preferred that the B-Raf gene is under the control of an inducible promoter. It is further preferred that the viable cell is a brain cell or a cell derived from a brain cell such as a cell from a brain cell line. Suitable cell lines include, e.g. CRL-11179, CRL-1074″, CRL-2299, CRL10442 or CCL-131 (ATCC numbers; cell lines available at www.atcc.org). Viable cells can also be derived from tissue samples of brain, spinal chord or, in the case of lymphocytes which are also preferred, from a blood sample or spleen sample and subsequently be cultured as primary cell culture. Suitable cell lines for lymphocytes are for example, HB-10569, HB-10220, CRL-8131 (ATCC numbers; cell lines are also available at www.atcc.org).

With all embodiments of the method of the invention including embodiments that make use of a viable cell, it is also preferred that the identification process is effected in a high throughput format. High-throughput assays, independently of being biochemical, cellular or other assays, generally may be performed in wells of microtiter plates, wherein each plate may contain 96, 384 or 1536 wells. Handling of the plates, including incubation at temperatures other than ambient temperature, and bringing into contact of test compounds with the assay mixture is preferably effected by one or more computer-controlled robotic systems including pipetting devices. In case large libraries of test compounds are to be screened and/or screening is to be effected within short time, mixtures of, for example 10, 20, 30, 40, 50 or 100 test compounds may be added to each well. In case a well exhibits biological activity, said mixture of test compounds may be de-convoluted to identify the one or more test compounds in said mixture giving rise to said activity.

In another preferred embodiment of the method of the invention, the change of B-Raf kinase activity is the absence, presence, increase or decrease of said 8-Raf kinase activity.

Methods to measure kinase activity are well-known in the art and have been described hereinabove.

A change in kinase activity effected by a compound modulating an anxiety or depression disorder can lead to the absence or to the presence of kinase activity relative to kinase activity without the compound. Advantageously, the level of activity is less than 90%, more preferred less than 80%, 70%, 60% or 50% of the activity in the absence of the compound. Preferred are compounds lowering the activity down to less than 25%, more particularly less than 10%, even more particularly less than 5% and most preferred less than 1% of the activity in the absence of the compound. In alternative embodiments said change refers to an increase of at least 10%, 20%, 40%, 60%, 80%, 100%, 200%, 500% or 1000% relative to kinase activity in the absence of the compound.

In a further preferred method of the invention, said modulation of expression results in a higher amount or lower amount of B-Raf protein compared to the amount of B-Raf protein in the absence of said compound.

Methods to measure the expression of proteins are well known in the art and are described for example in “Molecular Cloning: A Laboratory Manual” by Sambrook et al. (Cold Spring Harbour Laboratory Press) or “Current Protocols in Molecular Biology” (Ausubel et al., Wiley and Sons, Inc).

A change in expression of the B-Raf gene according to the invention can lead to the absence or to the presence of expression relative to expression without the compound. Advantageously, the level of expression is less than 90%, more preferred less than 80%, 70%, 60% or 50% of the expression level in the absence of the compound. Preferred are compounds lowering the expression level down to less than 25%, more particularity less than 10%, even more particularity less than 5% and most preferred less than 1% of the activity in the absence of the compound. In alternative embodiments said change refers to an increase of at least 20%, 40%, 60%, 80%, 100%, 200%, 500% or 1000% relative to expression in the absence of the compound.

The methods for identifying compounds described above and below preferably comprise the use of a suitable control. The methods can thus further comprise as a control the measurement of anxiety or depression behaviour of a wild-type mouse in the absence and the presence of a compound to be screened; and/or measuring the anxiety or depression behaviour of a B-Raf conditional knockout mouse in the presence and absence of said compound. The level of anxiety or depression behaviour of the wild-type mouse in the presence of the compound may then be compared to the level of anxiety or depression behaviour of the wild-type mouse in the absence of the compound; and the level of anxiety or depression behaviour of the B-Raf conditional knockout mouse in the presence of the compound may be compared to the B-Raf conditional knockout mouse in the absence of the compound. If the level of anxiety or depression behaviour of the wild-type mouse in the presence of the compound is decreased as compared to the wild-type mouse in the absence of the compound, and the level of anxiety or depression behaviour of the B-Raf conditional knockout mouse in the presence of the compound is similar to the level exhibited by the knockout mouse in the absence of the compound, then the compound—potentially—specifically inhibits B-Raf. In the screening methods of the present invention, the level of anxiety or depression behaviour of the wild-type mice or the B-Raf conditional knockout mice can be determined using a variety of methods as described herein or known to those skilled in the art. Further, suitable in vitro controls may make use of cells derived from B-Raf conditional knockout mice expressing no B-Raf or cells lacking or partially lacking B-Raf.

In a most preferred embodiment of the method of the invention, the expression of the B-Raf gene is determined by measuring any one of B-Raf transcript level, B-Raf protein level or B-Raf kinase activity.

Methods to measure transcript level, protein level or kinase activity are well-known to the skilled person. Measurement of kinase activity has been described supra. The transcript levels or protein levels of B-Raf can be measured by any method known that can provide quantitative information regarding the levels to be measured. The methods preferably are highly sensitive and provide reproducible results. In particular, methods based upon the polymerase chain reaction such as real-time PCR and related amplification technologies, such as NASBA and other isothermal amplification technologies, may be used. Further, microarray technique, immunoassay, western blotting are well-known basic methods, which can be applied. A suitable approach is, for example, real-time PCR employing the relative quantification approach to determine B-Raf transcript levels.

In another preferred embodiment, the method of the invention comprises a further step:
(c) administering the compound suspected to be capable of modulating an anxiety or depression disorder to a non-human animal and preferably non-human mammal and determining whether said compound modulates a B-Raf-mediated process relative to an untreated non-human animal, preferably non-human mammal,
   wherein the B-Raf-mediated process is selected from the group consisting of phosphorylation of intracellular or membrane proteins, maintenance of cellular membrane potentials or maintenance of anxiety behaviour and of depression behaviour.

Administration of compounds found to modulate anxiety or depression can be achieved with a variety of methods depending on the physical characteristics of the compound. Advantageously, the compound can be administered orally, but also other methods are encompassed, e.g. orally, topically, parenterally or by inhalation.

A non-human mammal can be for example, a rat, hamster, dog, monkey, rabbit, pig, goat or cow and preferably a mouse.

In another preferred embodiment of the method of the invention, the modulation of a B-Raf-mediated process results in a decrease of Erk1 and/or Erk2 protein activity.

The ability of the compound to modulate an anxiety or depression disorder can further be determined by detecting modulation of B-Raf mediated processes. Such processes can include, for example, biochemical processes (e.g., protein phosphorylation), or cellular processes (e.g., membrane potential) or behavioural processes, (e.g., anxiety or depression behaviour). The involvement of B-Raf in signaling cascades has been described in detail above and the skilled person will be able without further ado to determine suitable endpoints and methods for direct and indirect analysis of B-Raf function.

As Raf kinases phosphorylate and activate Mek kinases which in turn activate Erks, the latter are preferably suitable endpoints for studying modulation of a B-Raf-mediated process according to the invention. Any other molecule normally known to be directly or indirectly affected by B-Raf activity can be used as possible endpoint for said analysis, such as for example the Mek1 and Mek2 kinases. Studying modulation of suitable endpoints can, for example include measurement of phosphorylation rate and/or status, of the amount of protein, of gene expression levels, inter alia. Suitable methods include, for example, western-blotting, real-time PCR, and kinase-activity assays.

In a further preferred embodiment of the method of the invention, the compound is an inhibitor of B-Raf kinase activity or B-Raf gene expression.

The availability of B-Raf conditional knockout cells and mice facilitates the genetic dissection of B-Raf-mediated signalling pathways and allows for the identification of B-Raf specific inhibitors. For example, a compound that inhibits a function of B-Raf equally in a conditional knockout cell line and its wild-type parental cell line would be recognised as a non-B-Raf-specific inhibitor, while a compound that inhibits a B-Raf function in a wild-type cell line and has no effect in the conditional knockout cell line, would be recognized as a B-Raf specific inhibitor.

The term “inhibitor” designates an organic or anorganic compound lowering or abolishing the activity of a target molecule, preferably by performing preferably one or more of the following effects: (i) the transcription of the gene encoding the protein to be inhibited is lowered or abolished, (ii) the translation or stability of the mRNA encoding the protein to be inhibited is lowered or abolished, (iii) the protein performs its biochemical function with lowered efficiency or does not function at all in the presence of the inhibitor, and (iv) the protein performs its cellular function with lowered efficiency or does not perform at all in the presence of the inhibitor.

Compounds falling in class (i) include compounds interfering with the transcriptional machinery and/or its interaction with the promoter of said gene and/or with expression control elements remote from the promoter such as enhancers. Compounds of class (ii) comprise antisense constructs and constructs for performing RNA interference well known in the art (see, e.g. Zamore (2001) or Tuschl (2001)), preferably siRNA and shRNA constructs. Compounds of class (iii) interfere with molecular function of the protein to be inhibited, in the case of B-Raf with its enzymatic activity, in particular with the kinase activity. Accordingly, active site binding compounds, in particular compounds capable of binding to the active site of said protein kinase, are envisaged. More preferred are compounds specifically binding to an active site of B-Raf, for example, antibodies. The term “antibodies” comprises poly- and monoclonal antibodies, also derivatives or fragments thereof which still retain the binding specificity. Also encompassed are embodiments such as chimeric, single chain and humanized antibodies, as well as antibody fragments, like, inter alia, Fab fragments. Antibody fragments or derivatives further comprise F(ab′)₂, Fv or scFv fragments. Techniques for the production of antibodies, derivatives or fragments are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999.

In an additional embodiment small molecules are envisaged, which can be obtained by screening existing libraries as described supra, by buying commercially available products or by manufacturing the small molecules with methods well-known in the art. Also envisaged are compounds binding to or blocking substrate binding sites of B-Rat as are compounds binding to or blocking binding sites of B-Raf for other interaction partners. The latter group of compounds blocking binding sites of B-Raf may be fragments or modified fragments with improved pharmacological properties of the naturally occurring binding partners. Further envisaged are also B-Raf kinase destabilizers. Class (iv) includes compounds which do not necessarily directly bind to B-Raf, but still interfere with B-Raf activity, for example by binding to and/or inhibiting the function or inhibiting expression of members of a pathway which comprises B-Raf. These members may be either upstream or downstream of B-Raf within said pathway.

As mentioned, the inhibitor can be a small molecule, i.e. a low molecular weight compound. Low molecular weight compounds are compounds of natural origin or chemically synthesized compounds, preferably with a molecular weight between 100 and 1000, more preferred between 200 and 750, and even more preferred between 300 and 600.

The efficiency of the inhibitor can be quantified by comparing the level of activity in the presence of the inhibitor to that in the absence of the inhibitor. For example, as an activity measure may be used: the change in amount of mRNA formed, the change in amount of protein formed, the change in amount of substrate converted or product formed, and/or the change in the cellular phenotype or in the phenotype of an organism.

An inhibitor in accordance with the invention is aimed at alleviating the symptoms of an anxiety or depression disorder in a patient. Advantageously, the symptoms are completely abolished, but alternatively also a decrease in the severity of symptoms, in the quantity of symptoms and in the duration inter alia is envisaged in accordance with the invention. Methods to determine alleviation of symptoms are well known to the person skilled in the art. Alternatively, the inhibitor serves as a lead compound for developing a drug, according to conventional methods established in pharmacology. Such methods for the optimization of the pharmacological properties of compounds identified in screens, generally referred to as lead compounds, are known in the art and comprise a method of modifying a compound identified as a lead compound to achieve: (i) modified site of action, spectrum of activity, organ specificity, and/or (ii) improved potency, and/or (iii) decreased toxicity (improved therapeutic index), and/or (iv) decreased side effects, and/or (v) modified onset of therapeutic action, duration of effect, and/or (vi) modified pharmacokinetic parameters (resorption, distribution, metabolism and excretion), and/or (vii) modified physico-chemical parameters (solubility, hygroscopicity, color, taste, odor, stability, state), and/or (viii) improved general specificity, organ/tissue specificity, and/or (ix) optimized application form and route by (i) esterification of carboxyl groups, or (ii) esterification of hydroxyl groups with carboxylic acids, or (iii) esterification of hydroxyl groups to, e.g. phosphates, pyrophosphates or sulfates or hemi-succinates, or (iv) formation of pharmaceutically acceptable salts, or (v) formation of pharmaceutically acceptable complexes, or (vi) synthesis of pharmacologically active polymers, or (vii) introduction of hydrophilic moieties, or (viii) introduction/exchange of substituents on aromates or side chains, change of substituent pattern, or (ix) modification by introduction of isosteric or bioisosteric moieties, or (x) synthesis of homologous compounds, or (xi) introduction of branched side chains, or (xii) conversion of alkyl substituents to cyclic analogues, or (xiii) derivatisation of hydroxyl group to ketales, acetales, or (xiv) N-acetylation to amides, phenylcarbamates, or (xv) synthesis of Mannich bases, imines, or (xvi) transformation of ketones or aldehydes to Schiff's bases, oximes, acetates, ketales, enolesters, oxazolidines, thiazolidines or combinations thereof.

Advantageously, the level of activity is less than 90%, more preferred less than 80%, 70%, 60% or 50% of the activity in the absence of the inhibitor. Preferred are inhibitors lowering the level down to less than 25%, more particularity less than 10%, even more particularity less than 5% and most preferred less than 1% of the activity in the absence of the compound.

In a most preferred embodiment of the method of the invention, the inhibitor is selected from the group consisting of an antibody, siRNA, shRNA and a small molecule.

In another preferred embodiment of the method of the invention, the composition containing a viable cell comprising said B-Raf protein or said B-Raf gene in an expressible form is mounted on a solid support.

The term “solid support” as used herein refers to a flexible or non-flexible support that is suitable for mounting said composition or parts thereof comprising the B-Raf component. Said solid support may be homogenous or inhomogeneous. For example, said solid support may consist of different materials having the same or different properties with respect to flexibility and immobilization, for instance, or said solid support may consist of one material exhibiting a plurality of properties also comprising flexibility and immobilization properties. The solid support according to the invention provides a surface for the attachment of the compositions or parts thereof comprising the B-Raf component or compounds identified in accordance with the invention. The surface may be a coating applied to the support or carrier, or the surface of the support or carrier itself may be used. Support or carrier materials commonly used in the art and comprising glass, plastic, gold and silicon are envisaged for the purpose of the present invention. Coatings according to the invention, if present, include poly-L-lysine- and amino-silane-coatings as well as epoxy- and aldehyde-activated surfaces.

The term “mounted” means that the molecular species of interest is fixed to a solid support, preferably covalently linked thereto. This covalent linkage can be achieved by different means depending on the molecular nature of the molecular species. Moreover, the molecular species may be also fixed on the solid support by electrostatic forces, hydrophobic or hydrophilic interactions or Van-der-Waals forces. The above described physico-chemical interactions typically occur in interactions between molecules. For example, biotinylated polypeptides may be fixed on a avidin-coated solid support due to interactions of the above described types. Further, proteins such as antibodies, may be fixed on an antibody coated solid support. Moreover, the immobilization is dependent on the chemical properties of the solid support. For example, nucleic acid molecules can be immobilized on a membrane by standard techniques such as UV-crosslinking or heat.

In accordance with the foregoing, in a most preferred embodiment, the solid support is a membrane, a glass-, polypropylene- or silicon-chip, are beads or a bead array.

In a most preferred embodiment of the method of the invention, said cell is part of a tissue.

In this preferred aspect of the invention the composition comprising B-Raf protein can be a tissue. The tissue consists of cells that can naturally express B-Raf or be transiently or stably transfected with a B-Raf expression vector to express said protein in detectable amounts and function within the cell. Design, manufacture, transfection, protein expression and isolation are methods well-known in the art and described for example in “Molecular Cloning: A Laboratory Manual” by Sambrook et al. (Cold Spring Harbour Laboratory Press). It is particularly preferred that said tissue is a non-human brain tissue such as a non-human primate brain tissue. Further, it is particularly preferred that said tissue is a non-human spinal chord tissue such as a non-human primate spinal chord tissue.

In a further preferred embodiment of the method of the invention, said compound can cross the blood-brain barrier.

Advantageously, the compound identified according to the above method of the invention will naturally be able to cross the blood-brain barrier. Nevertheless, compounds can also be modified to allow for crossing of said barrier. Methods to enable drug targeting in the brain are well-known in the art and include, for example, disruption of the barrier by osmotic means, use of vasoactive substances (e.g. bradykinin), localized high intensity focused ultrasound (HIFU), endogenous transport systems like glucose and amino acid carriers, receptor-mediated transcytosis, liposome-mediated passage, brain injection, intracerebral implantation and convection-enhanced distribution.

In another preferred embodiment of the method of the invention, the modulation of an anxiety or depression disorder is a reduction of the severity of symptoms or the absence of symptoms associated with said anxiety or depression disorder.

Advantageously, the compound identified according to the method of the invention modulates the anxiety or depression disorder with the effect of a reduction of the severity or even complete abolishment. Methods to determine reduction of symptoms are well known to the person skilled in the art and guidance is provided throughout the specification. It is generally envisaged that the reduction which is most advantageously an abolishment of symptoms is achieved by using the inhibitor discussed hereinabove or a drug derived from said inhibitor wherein the inhibitor is used as lead compound.

Advantageously, the severity of the symptoms is less than 90%, more preferred less than 80%, 70%, 60% or 50% of the severity in the absence of the compound. Preferred are compounds reducing the severity down to less than 25%, more particularity less than 10%, even more particularity less than 5% and most preferred less than 1% of the severity in the absence of the compound.

In a further embodiment, the present invention relates to a method of treating an anxiety or depression disorder in an individual comprising administering to the individual an effective amount of a compound that inhibits B-Raf kinase activity or inhibits expression of the B-Raf gene.

As mentioned, the present invention is based on the finding that B-Raf activity in neurons of the forebrain mediates processes involved in anxiety and depression behaviour. The rationale for using a B-Raf inhibitor to treat patients with anxiety or depression disorder lies in the finding that B-Raf knockout mice revealed antidepressive and a strongly reduced anxiety related behaviour. Thus, it is expected that the symptoms of said patients will be alleviated combined with an increase in quality of life upon treatment with said B-Raf activity inhibiting compounds. Any compound that is known or preferably identified by the methods of the present invention to inhibit B-Raf activity will be suitable as an agent for treatment or as a lead compound for developing such an agent. Drug formulation, ways of administration and dosage regimen are detailed elsewhere in this specification and apply mutatis mutandis to the method of treatment.

The term “effective amount” is, e.g., an amount that inhibits, abolishes or reduces the activity or expression of B-Raf, and results in a significant, e.g., a statistically significant difference, e.g. decrease, in a cellular or behavioural function that is normally subject to regulation, e.g., a positive regulation by B-Raf. For example, an effective amount of a therapeutic compound administered to an individual would comprise an amount sufficient to alter (inhibit) B-Raf mediated protein phosphorylation and thereby decrease the level of anxiety or depression behaviour. The amount of compound required to inhibit B-Raf activity will vary depending on a variety of factors including the size, age, body weight, general health, sex, and diet of the individual as well as the time of administration, and the duration or stage of the particular condition or disease that is being treated. Effective dose ranges can be extrapolated from dose-response curves derived from an in vitro or an in vivo test system.

In another embodiment, the present invention relates to the use of a compound that inhibits B-Raf kinase activity or expression of the B-Raf gene in the manufacture of a pharmaceutical composition for treating an anxiety or depression disorder.

In an alternative embodiment the invention relates to a compound that inhibits B-Raf kinase activity and B-Raf gene expression in treating an anxiety or depression disorder. Preferably, said compound is an inhibitor of B-Raf activity or B-Raf gene expression. The compound will usually be formulated into a pharmaceutical composition.

The pharmaceutical composition may conveniently be administered by any of the routes conventionally used for drug administration, for instance, orally, topically, parenterally or by inhalation. For example, injection of the pharmaceutical composition and subsequent absorption into the blood circulation allows for transport to the brain capillaries where it can cross the blood brain barrier according to a suitable of the above methods, for example liposome-mediated passage. The compound may be administered in conventional dosage forms prepared by combining the drugs with standard pharmaceutical carriers and/or additional substances aimed at facilitating crossing the blood brain barrier according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables. The carrier(s) must be “acceptable” in the sense of being compatible with the other ingredients of the formulation and not deleterious to the recipient thereof. The pharmaceutical carrier employed may be, for example, either a solid or liquid. Exemplary of solid carriers are lactose, terra alba, sucrose, talc, gelatin, agar, pectin, acacia, magnesium stearate, stearic acid and the like. Similarly, the carrier of diluent may include time delay material well known to the art, such as glyceryl mono stearate or glycerol distearate alone or with a wax.

The dosage regimen will be determined by the attending physician and other clinical factors; preferably in accordance with any one of the above described methods. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment.

In a further preferred embodiment of the method or the use of the invention, the compound is selected from the group consisting of Nexavar/BAY 43-9006/Sorafenib, CHIR-265, X-6-(3 acetamidophenyl) pyrazines, 3,5, Di-substituted pyridines, SB-590885 (33), AAL881, LBT613, Omega-carboxypyridyl, Compound 2, ZM 336372, L-779450, PLX4032, 17-allylamino-17-demethoxygeldanamycin, 17-DMAG, ISIS 5132, LErafAON-ETU, SAHA and NVP-LAQ824.

Presently, efforts are made to develop modulators, preferably inhibitors of Raf kinases, especially B-Raf kinase inhibitors due to their recently appreciated Influence in tumorigenesis. The modulators or inhibitors have partially entered clinical trials in different phases. For example, CHIR-265 is in clinical phase I and Nexavar/BAY 43-9006/Sorafenib has even been approved and is currently used in the US, Mexico, Switzerland and Germany since 2005 and 2006, respectively. The compounds encompass small molecule inhibitors, such as for example, Nexavar/BAY 43-9006/Sorafenib, CHIR-265, antisense molecules, such as for example, ISIS 5132, LErafAON-ETU, Raf kinase destabilizers, such as for example, 17-DMAG, SAHA. Further information regarding Nexavar/BAY 43-9006/Sorafenib (Bayer/Onyx) can be found in Wright et al., Clinical trials referral resource. Oncology (Huntingt) 2005; 19:499-502. Information on CHIR-265 (Chiron) can be found in Tsai et al., Development of a novel inhibitor of oncogenic B-Raf. In the 97^{th} AACR annual meeting, Washington D.C., 2006. Abstract No 2412. Information on X-6-(3 acetamidophenyl) pyrazines (Center for Cancer Therapeutics, Sutton, UK) can be found in Niculescu-Duvaz et al., Novel inhibitors of B-Raf based on a disubstituted pyrazine scaffold. Generation of a nanomolar lead. J Med Chem 2006; 49:407-16.

Information on 3,5, Di-substituted pyridines (Center for Cancer Therapeutics, Sutton, UK) can be found in Newbatt et al., Identification of inhibitors of the kinase activity of oncogenic V600BRAF in an enzyme cascade high-throughput screen. J Biomol Screen 2006; 11:145-54. Information on SB-590885 (33) (GlaxoSmithKline) can be found in Takle et al., The identification of potent and selective imidazole-based inhibitors of B-Raf kinase. Bioorg Med Chem Lett 2006; 16:378-81. Information on AAL881 (Novartis) can be found in Ouyang et al., Inhibitors of Raf kinase block growth of thyroid cancer cells with RET/PTC or BRAF mutations in vitro and in vivo. Clin Cancer Res 2006; 12:1785-93. Information on LBT613 (Novartis) can be found in Khire et al., Omega-carboxypyridyl substituted ureas as Raf kinase Inhibitors: SAR of the amide substituent. Bioorg Med Chem Lett 2004; 14:783-6. Information on Omega-carboxypyridyl (Bayer) can be found in Lackey et al., The discovery of potent cRaf1 kinase inhibitors. Bioorg Med Chem Lett 2000; 10:223-6. Information on Compound 2 (GlaxoSmithKline) can be found in Hall-Jackson et al., Paradoxical activation of Raf by a novel Raf inhibitor. Chem Biol 1999; 6:559-68. Information on ZM 336372 (AstraZeneca) can be found in Heimbrook et al., Identification of potent, selective kinase inhibitors of Raf. Am Assoc Cancer Res 1998; 39:558.[Abstract No 3739]. Information on L-779450 (Merck) can be found in Hall-Jackson et al., Paradoxical activation of Raf by a novel Raf inhibitor. Chem Biol 1999; 6:559-68. Information on PLX4032 (Plexxikon) can be found in Venetsanakos et al., CHIR-265, a novel inhibitor that targets B-Raf an VEGFR, shows efficacy in a broad range of preclinical models. In the 97^{th} AACR annual meeting, Washington D.C., 2006. Abstract No 4854. Information on 17-allylamino-17-demethoxygeldanamycin can be found in Budillon et al., Multiple-target drugs: inhibitors of heat shock protein 90 and of histone deacetylase. Curr Drug Targets 2005; 6:337-51. Information on 17-DMAG can be found in Hollingshead et al., In vivo antitumor efficacy of 17-DMAG, a water-soluble geldanamycin derivative. Cancer Chemother Pharmacol 2005; 56:115-25. Information on ISIS 5132 can be found in Monia et al., Antitumor activity of a phosphorothioate antisense oligodeoxynucleotide targeted against C-Raf kinase. Nat Med 1996; 2:668-75. Information on LErafAON-ETU can be found in Gokhale et al., Pharmacokinetics, toxicity, and efficacy of ends-modified raf antisense oligodeoxyribonucleotide encapsulated in a novel cationic liposome. Clin Cancer Res 2002; 8:3611-21. Information on SAHA can be found in Mitsiades et al., Transcriptional signature of histone deacetylase inhibition in multiple myeloma: biological and clinical implications. Proc Natl Acad Sci USA 2004; 101:540-5. Information on NVP-LAQ824 can be found in Fuino et al., Histone deacetylase inhibitor LAQ824 down-regulates Her-2 and sensitizes human breast cancer cells to trastuzumab, taxotere, gemcitabine, and epothilone B. Mol Cancer Ther 2003; 2:971-84. Furthermore, additional modulators presently available but not here specified are also envisaged.

In a further embodiment, the present invention relates to a method of diagnosing a B-Raf-associated anxiety or depression disorder comprising the steps of:
(a) determining the level of B-Raf kinase activity or B-Raf gene expression in a sample obtained from a patient; and
(b) comparing the level of B-Raf kinase activity or B-Raf gene expression obtained in (a) with said levels in a control sample obtained from an individual not affected by a B-Raf-associated anxiety or depression disorder,
   wherein a change in the level of activity of the B-Raf kinase or of the expression of the B-Raf gene relative to the control sample is indicative of a B-Raf-associated anxiety or depression disorder.

A sample may be any cell or tissue which allows for studying B-Raf kinase activity levels. The samples and control samples are preferably to be obtained from the same compartment of the body and processed identically to exclude inter assay variability and guarantee meaningful results. A sample may be tissues or fluids containing cells, like for example blood, saliva, urine, lymph, neuronal tissue, serum, cerebrospinal fluid and skin.

As is evident to the person skilled in the art, the molecular knowledge deduced from the present invention can now be used to exactly and reliably diagnose the molecular cause of an anxiety or depression disorder in a patient as far as it is B-Raf related. Advantageously, an anxiety or depression disorder can even be predicted and preventive or therapeutic measures can be applied accordingly. Preventive and therapeutic measures are preferably based on the use of a compound known to inhibit B-Raf or a compound identified according to the methods of the invention. Moreover in accordance with the foregoing, in cases where a given drug takes an unusual effect, a suitable individual therapy can be designed based on the knowledge of the individual molecular levels of B-Raf activity of a subject with respect to therapeutics that are developed on the basis of compounds identified according to the methods of the invention.

In accordance with the foregoing, the sample is in a preferred embodiment of the method selected from the group comprising brain tissue, spinal chord tissue or lymphocytes.

In a preferred embodiment, the method comprises a further step:
(c) administering an effective amount of a compound that has been identified according to the method of the invention to a patient having a B-Raf-associated anxiety or depression disorder.

Due to the present invention it is now possible to identify and develop new drugs for anxiety or depression disorders and furthermore diagnose the latter in patients. The combination of these new insights further allows for a selective therapy to be chosen by the medical practitioner to treat patients with an anxiety or depression disorder. If a patient is diagnosed as having a B-Raf-associated anxiety or depression disorder, a suitable therapy can be applied according to the individual make up of the patient's B-Raf activity levels detected. This provides a therapy that displays reduced side-effects, a specific cause-related mode of action and a better long-term tolerance as compared to the presently used drugs for treating anxiety or depression disorders.

In another embodiment, the invention relates to a genetically engineered mouse transgenic for (a) a Cre recombinase gene operatively linked to a CamKIIα promoter and (b) a loxP site flanking each exon boundary of exon 12 of the B-Raf gene obtainable by crossing transgenic line CamKII-CRE-159 with transgenic line B-raf-flox.

The present invention provides a conditional knockout mouse established using the loxP/Cre recombinase system. The loxP/Cre recombinase system is well-known in the art and is further described and referenced in the example section of the specification. As used herein, conditional knockout refers to a genetically modified organism that has a genome, in which a particular gene has been disrupted or deleted such that expression of the gene is eliminated or occurs at a reduced level in a specific cell type or tissue (Kwan, Genesis, 32, 49-62 (2002)) (Rajewsky, et al., J Clin Invest, 98, 600-603 (1996)). The disruption or deletion of the particular gene, in this case the B-Raf gene, is based on the interaction of the following elements: loxP-sites in the B-Raf gene and Cre-Recombinase under the control of a tissue specific promoter. The transgenic, conditional knockout mouse of the invention lacks a functional B-Raf gene product or exhibits a reduced level of the B-Raf gene product in neurons of the forebrain. The mutant mouse is referred to hereinafter as a “conditional B-Raf knockout mouse” or “Braf^{flox/flox}/CamKII-cre mouse”.

The present invention also encompasses methods of producing a transgenic mouse that lacks a functional B-Raf gene in a conditional manner. Briefly, the standard methodology for producing a conditional knockout mouse is well known in the art (Kwan, Genesis, 32, 49-62 (2002)) (Rajewsky, et al., J Clin Invest, 98, 600-603 (1996)) and requires the crossing of an allele of the target gene, that has been modified by the insertion of two Cre recombinase recognition (loxP) sequences within intron regions (“floxed”), to a second mouse strain that expresses Cre recombinase in a specific cell type or tissue. By the action of Cre the loxP flanked gene segment is excised and deleted from the genome leading to the inactivation of the B-Raf gene.

The present transgenic mouse has been generated by crossing the transgenic mouse line B-raf-flox in which exon 12 of the B-Raf gene is flanked by two loxP sequences (cf. FIG. 1) described and manufactured by Chen, et al. (J Neurosci Res, 83, 28-38 (2006)) to the transgenic mouse line CamKII-CRE-159 that expresses Cre recombinase under the control of the CamKIIα promoter described and manufactured by Minichiello, et al. (Neuron, 24, 401-414 (1999)). The thus obtained transgenic mouse of the invention displays a superior deletion profile of B-Rat compared to other B-Raf conditional knockout mice (cf. Chen, et al., J Neurosci Res, 83, 28-38 (2006)). The use of the CamKII-CRE-159 mouse line led already at an age of about 8 weeks to about 50% recombination in hippocampus, cortex and olfactory bulb, which consist only of about 50% of CamKII expressing neurons, thus resembling a maximum of recombination efficiency.

As a result of the conditional disruption of the B-Raf gene, the B-Raf conditional knockout mouse of the present invention manifests a particular phenotype. The term phenotype refers to the resulting biochemical, physiological or behavioural consequences attributed to a particular genotype. In the situation where a conditional knockout mouse has been created, the phenotype observed is a result of the loss of the gene that has been knocked out. In one embodiment, the B-Raf conditional mutant mouse exhibits reduced anxiety and depression behaviour when compared to a wild type mouse in specific tests for the measurement of anxiety or depression behaviours. Such transgenic animals are well suited for, e.g., pharmacological studies of drugs.

The B-Raf knockout mice described herein can also be bred (e.g., inbred, outbred, or crossbred) with appropriate mates to produce colonies of animals, whose genomes comprise at least one non-functional allele of the endogenous gene that naturally encodes and expresses functional B-Raf. Examples of such breeding strategies include, but are not limited to: crossing of heterozygous conditional knockout animals to produce homozygous conditional animals; outbreeding of founder animals (e.g., heterozygous or homozygous conditional knockouts) with a mouse that provides an animal model of an anxiety or depression disorder; and crossbreeding a founder animal with an independent transgenic animal that has been genetically engineered to overexpress a gene associated with increased susceptibility to anxiety- and/or depression-related behavior.

A method of identifying another gene contributing to the pathophysiology of an anxiety or depression disorder apart from B-Raf represents a further embodiment of the invention, said method comprising the steps of:
(a) crossing the genetically engineered mouse of the invention with mice known to harbour mutations in other signaling pathways;
(b) determining the contribution of said signaling pathways in the regulation of anxiety and depression behaviour.

In accordance with this embodiment of the invention, the knowledge provided by the present invention can also be used to further elucidate the contribution of B-Raf or the contribution of other signaling pathways to the etiology and pathophysiology of anxiety or depression disorders. The transgenic mouse of the invention can be crossed to other transgenic mice and their anxiety and depression behaviour can be examined, for example by the methods provided in this specification. The other transgenic mouse can harbour preferably one, but also more mutations that either lead to an increase or decrease, presence or absence of a gene product as compared to the unmanipulated mouse. A change in anxiety or depression related behaviour of said mouse as compared to the B-Raf mouse is indicative for the involvement of the gene product of the mutated gene which has been introduced in addition to the mutant B-Raf gene in anxiety or depression disorders. This change can be an increase, decrease, absence or presence of anxiety or depression behaviour. The comparison of said change and the extent of that change can be used to assess the involvement of other genes in the etiology and pathophysiology of anxiety and depression disorders and hence be basis for the classification into “negative” or “positive” modulator. This method can provide further insights and reveal new drug targets for a therapeutical approach to anxiety or depression disorders. The person skilled in the art is well-aware of mice carrying mutation(s) in relevant genes and is in the position to manufacture mice by crossing of the B-Raf^{flox/flox}/CamKII-cre mouse line and the mouse line carrying a mutation. Further, methods to test anxiety behaviour or depression behaviour and assess changes in the latter are well-known and are further provided in this specification.

In another preferred embodiment of the method or the use of the invention, the anxiety or depression disorder is selected from the group consisting of generalized anxiety disorder, social phobia, simple phobia, panic disorder, post-traumatic stress disorder (PTSD), obsessive-compulsive disorder (OCD), major depression disorder, dysthymic disorder, bipolar I disorder, bipolar II disorder, cyclothymic disorder, and depressive disorder not otherwise specified.

As the present invention is based upon a novel and fundamental finding it can easily be envisioned that the majority of presently known anxiety and depression disorders can be treated by compounds modulating B-Raf activity, expression and B-Raf mediated processes. Furthermore, most anxiety and depression disorders are currently treatable with the same class of drugs which suggests a close relationship and common molecular mechanism of said anxiety and depression disorders. In general, anxiety disorders are characterized by a specific or general increase of anxiety behaviour and depressive disorders are characterized by a specific or general increase of depressive behaviour.

Various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

The figures show:

FIG. 1. Experimental Scheme to Generate B-Raf Conditional Knockout Mice by Crossing Braf-Flox with CamKII-Cre Mice.

Scheme of the floxed B-Raf allele before (A) and after Cre recombination (B). Exon 12 is flanked by loxP sites (lox) and excised by Cre recombinase (Cre), resulting in a null mutation. The protein structure (C) shows Exon 12 (red) at the start of the kinase domain. D: PCR-genotyping with the primers Braf_{—}9, Braf_{—}11, and Braf_{—}17 was used to determine wild-type (wt, 357 bp), floxed (flox, 413 bp), and deleted (del, 282 bp) alleles. Ras-BD: Ras-binding domain; Cys: cystein-rich domain; CR1-3: conserved regions of RAF proteins; N: amino terminus; C: carboxyl terminus; Ø: negative control.

FIG. 2. Demonstration of the Forebrain-Specific B-Raf Knockout.

A: PCR detection of floxed (flox) and excised (del) Exon 12 from Braf-flox mice. No recombination occurred in B-Raf^{flox/flox} (flox/flox) mice, whereas the deleted allele is visible mainly in forebrain regions of B-Raf^{flox/flox}/CamKII-cre (Δ/Δ) mice. B: Western blot against B-Raf protein on brain regions of mutant B-Raf^{flox/flox}/CamKII-cre mice (Δ/Δ) and control B-Raf^{flox/flox} mice (flox/flox). An antibody against β-ACTIN was used as loading control. OB: olfactory bulb; HC: hippocampus; St: striatum; fCx: Cortex, frontal part; pCx: Cortex, posterior part; Th: thalamus; MB: midbrain; Cb: cerebellum; BS: brainstem; Ø: negative control.

FIG. 3. Demonstration of the Loss of Downstream MAPK Signalling in B-Raf Conditional Knockout Mice.

A: Western blotting of protein from hippocampus of B-Raf^{flox/flox} control mice (flox/flox) and B-Raf^{flox/flox}/CamKII-cre mutant mice (Δ/Δ) shows the loss of B-Raf protein in mutants. Reduction of Erk1/2 phosphorylation (pERK1/2) is shown in the basal as well as in the activated state of mutant mice. An antibody against total Erk1/2 detects an equal amount of protein in both genotypes and activation levels. B: Immunohistochemistry for phosphorylated Erk1/2 shows protein expression in the hypothalamus of control (flox/flox) and mutant (Δ/Δ) mice following foot shock (activated) or control treatment (basal). Scale bars in B: 2.5 mm.

FIG. 4. Reduced Anxiety Behaviour of B-Raf Conditional Knockout Mice in the Light-Dark Exploration Test.

Total duration (A), number of entries (B), distance traveled (C), and number of turns in the light compartment (D) are shown for mutant B-Raf^{flox/flox}/CamKII-cre mice (flox/flox) and control animals (Δ/Δ). Males and females are shown in a pooled representation, since no sex specific effect was observed. *: p<0.05, ***: p<0.001.

FIG. 5. Reduced Anxiety Behaviour of B-Raf Conditional Knockout Mice in the Elevated Plus Maze Test.

Total duration (A) and number of entries in the open arms of the maze (B), number of entries in the closed arms (C), and total distance traveled in the open arms (D) are shown for mutant B-Raf^{flox/flox}/CamKII-cre mice (flox/flox) and control animals (Δ/Δ). Males and females are shown in a pooled representation, since no sex specific effect was observed. **: p<0.01, ***: p<0.001.

FIG. 6. Antidepressant Behaviour of B-Raf Conditional Knockout Mice in the Forced Swim Test.

Time spent swimming (A/D), floating (B/E), and struggling (C/F) during the 6 min test phase are shown for mutant B-Raf^{flox/flox}/CamKII-cre mice (flox/flox, black) and control animals (Δ/Δ, white/green). Total times for the three recorded behaviour types are depicted in A-C, and activities in 1 min intervals over the whole test phase are given in D-F. Males and females are shown in a pooled representation in A, D, E, and F, since no sex specific effect was observed. In B and C, data for both sexes are shown separately (males in blue and females in red). n.s.: not significant; *: p<0.05, **: p<0.01, ***: p<0.001.

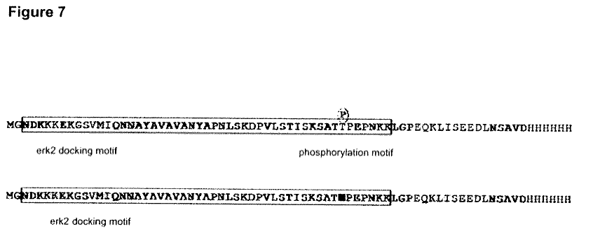

FIG. 7. Oligonucleotides for In Vitro Kinase Assay

Depicted are two 75 aa long oligopeptides, one with the correct sequence of amino acids 351-399 of the α2 subunit of the GABA_{A} receptor and another one with the same sequence but with a mutated phosphorylation site (T393V).

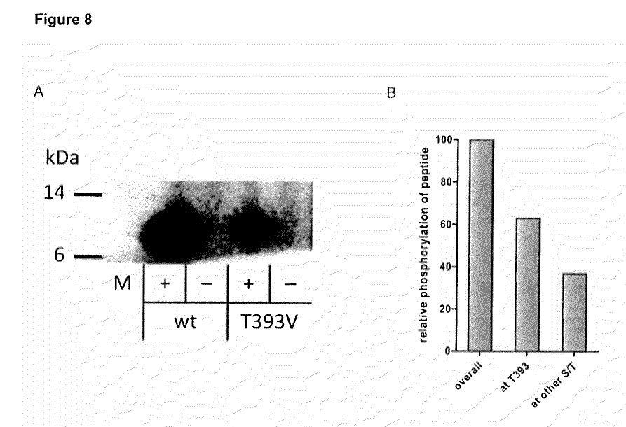

FIG. 8. In Vitro Kinase Assay—Measurement of Phosphorylation

In FIG. 8A, the incorporation of ³²P as a readout of phosphorylation is measured. The construct with the wildtype sequence shows a much higher incorporation of ³²P than the second construct with the mutated T393. This demonstrates that the Erk2 kinase has indeed a higher bias for phosphorylating this site in vitro. The background phosphorylation of the mutated construct can be explained by unspecific phosphorylation at all serine/threonine residues due to the excess of Erk2 in the reaction. Comparing the amounts of incorporated ³²P at T393 and at the other Ser/Thr leads to the conclusion that 63% of total phosphorylation occurs at the putative Erk2 site, whereas the other nine Ser/Thr residues are phosphorylated at an average of 4% each (FIG. 8B).

The examples illustrate the invention:

### EXAMPLE 1

### B-Raf Conditional Mutant Mouse Design, Breeding and Genotyping, Immunoblotting and Immunohistochemistry

To generate B-Raf conditional mutant mice, in which the B-Raf gene is specifically inactivated in neurons of the forebrain, the Braf-flox mouse strain in which exon 12 of the B-Raf gene is flanked by two loxP sequences (FIG. 1) (Chen, et al., J Neurosci Res, 83, 28-38 (2006)), was crossed to a CamKII-cre transgenic mouse strain (Minichiello, et al., Neuron, 24, 401-414 (1999)) that expresses Cre recombinase under the control of the CamKIIα promoter. This experimental strategy restricts the inactivation of the B-Raf gene to neurons of the forebrain (cortex, hippocampus, amygdala, striatum, offactory bulb) (FIG. 2) and to postnatal development and thereby circumvents the embryonic lethality associated with the complete germline inactivation of the B-Raf gene. Since B-Raf is only expressed in neurons the B-Raf protein e.g. in the hippocampus is undetectable in B-Raf conditional knockout mice and the reduced level of activated Erk1 and Erk2 kinases demonstrates the loss of downstream MAPK signalling (FIG. 3). The behaviour of adult B-Raf conditional mutants (B-Raf^{flox/flox}/CamKII-cre) was compared side by side to age matched littermate control mice of the B-Raf^{flox/flox} genotype that contain two copies of the loxP modified, functional B-Raf gene. The level of anxiety behaviour of mutant and control mice was compared in the light/dark exploration test (FIG. 4) and the elevated plus maze test (FIG. 5), while the level of depression behaviour was assessed in the forced swim test (FIG. 6).

A. Mouse Genotyping and Breeding

In the mouse line Braf-flox, exon 12 of the B-Raf gene, which is the first exon encoding the kinase domain of the B-Raf protein, is flanked by loxP sites (FIG. 1A). For this modification, a targeting vector, containing a 1.2 kb fragment flanking exon 12, the loxP sites, and a neomycin selection marker, was inserted into one B-Raf allele by homologous recombination in ES cells. The neomycin selection marker was deleted in a later stage of ES cell culture. After the modification, the allele encodes the active B-Raf protein, but can be inactivated by Cre recombinase mediated deletion of the sequence between the two loxP sites (Chen, et al., J Neurosci Res, 83, 28-38 (2006)). The deletion of the floxed exon by Cre recombination results in a shift in the open reading frame and therefore in a null mutation of the B-Raf gene (FIGS. 1B and C).

For genotyping of the wild-type, floxed, and deleted B-Raf alleles, a triplex PCR to distinguish the wild-type allele from the floxed and deleted alleles was performed with the following primers: Braf_{—}9 (SEQ ID NO:5), Braf_{—}11 (SEQ ID NO:6), and Braf_{—}17 (SEQ ID NO:7) wild-type. In the wild-type allele, primer Braf_{—}9 and Braf_{—}11 amplified a 357 by fragment in intron 11. Due to one of the inserted loxP sites in the floxed allele, the fragment enlarged to 413 by in this case. After Cre mediated deletion of exon 12, the binding site of primer Braf_{—}11 was lost, but primer Braf_{—}9 and Braf_{—}17 amplified a 282 by fragment (FIG. 6D). Upon crossing the Braf-flox mice to mice expressing Cre recombinase from the Ca²⁺/calmodulin-dependent protein kinase II a (CamKIIα) promoter (Minichiello, et al., Neuron, 24, 401-414 (1999)), deletion of exon 12 occurred specifically in the forebrain of double transgenic offspring, as shown in FIG. 2. For genotyping of the CamKII-cre transgene a PCR was performed with the primers pCre1 (SEQ ID NO:8) and pCre2 (SEQ ID NO:9); the presence of the Cre transgene is indicated by a 447 by amplification product.

Braf-flox mice were received on a FVB background and were backcrossed for three generations to C57Bl/6J. Generally, they were group housed in open cages. Mice for the behavioural analyses were housed in individually ventilated cages from the age of 8-10 weeks on.

B. Immunoblotting and Immunohistochemistry

Total protein was extracted from brain tissue. Tissue was homogenized in RIPA buffer (50 mM Tris-HCl pH 7.4, 1% NP-40, 0.25% sodiumdesoxycholat, 150 mM NaCl, 1 mM EDTA, protease inhibitor), sonificated and centrifuged. 50 μg protein of each sample were run on a 10% Tris-HCl gel (Biorad) and blotted on a PVDF membrane (Pall). After blocking with 4% skim milk (5% BSA for phosphoproteins) the membrane was incubated with the first antibody (3 hours or overnight), washed with TBST, incubated with the second horseradish-peroxidase-conjugated antibody (1 hour) and washed with TBST. The detection reaction was initiated with ECL detection reagents (Amersham) and the membrane was exposed to Hyperfilm (Amersham). The antibodies used for Western blotting were anti-b-Actin (AC-15, #ab6276, Abcam, 1:100,000), anti-B-Raf (sc-166, Santa Cruz Biotechnology, 1:600), anti-pERK1/2 (#9101, Cell Signaling Technology, 1:1,000), anti-Erk1/2 (#9102, Cell Signaling Technology, 1:1,000), anti-mouse (Dianova, 1:1,000), and anti-rabbit (Dianova, 1:5,000).

Activation of MAPK signalling was achieved by application of a mild foot shock. Mice were placed in startle boxes (Med Associates Inc., Startle Stimulus Package PHM-255A, ANL-925C Amplifier) and after a 5 min accommodation interval, ten foot shocks (0.5 sec, 0.4 mA) were applied to the animals, interrupted by variable inter-trial intervals of 180-330 sec. Control mice were subjected to the same context and procedure, but without receiving the foot shocks. Animals were put back in their home cages and they were killed 60 min after the end of the program.

For histology, mice were rapidly anesthetized with CO₂ and perfused intracardially for 5 min with ice-cold 4% paraformaldehyde (PFA) in 0.1 M Na₂HPO₄/NaH₂PO₄ buffer, pH 7.5 (PBS). Brains were dissected, post-fixed in 4% PFA/PBS for 24 hr at 4° C., and incubated in 25% sucrose/PBS for 24 hr at 4° C. for cryoprotection. Sections (30 μm) were cut on a cryostat (Leica) and stored in a solution containing 30% ethylene glycol and 30% glycerol in PBS at −20° C. until processing. For immunohistochemistry, free-floating sections were rinsed overnight in Tris-buffered saline (TBS; 0.05 M Tris and 0.15 M NaCl, pH 7.5). Endogenous peroxidase was quenched by incubation of the sections for 5 min in TBS containing 3% H₂O₂ and 10% methanol. Sections were then rinsed three rimes for 10 min each in TBS. Cell membranes were permeabilized by incubation for 15 min in 0.5% Triton X-100 in TBS. After three washes for 5 min each in TBS, sections were incubated overnight with the first antibody in TBS at 4° C. After three rinses in TBS sections were incubated for 2 hr at room temperature with the secondary biotinylated antibody in TBS. After three washes for 5 min each in TBS, the sections were incubated for 60 min in avidin-biotin-peroxidase complex (ABC) solution (Vector Laboratories, 1:300). The sections were then washed once in TBS and twice in TB (0.05 M Tris, pH 7.5) for 10 min each, placed in a solution of TB containing 0.1% 3,3′-diaminobenzidine (DAB; 50 mg/100 ml), and developed for 30 min after addition of 0.02% H₂O₂. The reaction was stopped by washing the sections three times in TB. The tissue sections were mounted onto poly-L-lysine-coated slides, air-dried and dehydrated through alcohol to xylene for light microscopic examination. The antibodies used for immunohistochemistry are anti-pERK1/2 (#9101, Cell Signaling Technology, 1:400) and goat anti-rabbit (Dianova, 1:200).

### EXAMPLE 2

### Behavioural Analyses and Data Processing

For behavioural analyses, groups of 10-15 male and female animals at the age of 3-6 months with a maximal age difference of two weeks within the groups were used. For the Light-Dark exploration test, the test box was made of PVC and divided into two compartments, connected by a small tunnel (4×6×9 cm high). The lit compartment (29×19×24 cm high) was made of white PVC and was illuminated by cold light with an intensity in the centre of 650 lux. The dark compartment (14×19×24 cm high) was made of black PVC and not directly illuminated (approx. 20 lux in the centre). The mouse was placed in the centre of the dark compartment and allowed to freely explore the apparatus for 5 min. Behaviours were observed by a trained observer sitting next to the box using a hand-held computer. Data were analyzed with respect to (1) the number of entries, latency to first entry, and time spent in both compartments and the tunnel; and (2) the number of rearings in both compartments and the tunnel. An entry into a compartment was defined as placement of all four paws into the compartment. Additionally, a camera was mounted above the center of the test arena to videotape the trial, and the animal's locomotor path in the lit compartment was analyzed with a video-tracking system. The box was cleaned before each trial with a disinfectant.

The test arena for the elevated plus maze test was made of light grey PVC and consisted of two open arms (30×5×0.3 cm) and two closed arms of the same size with 15 cm high walls. The open arms and accordingly the closed arms were facing each other connected via a central square (5×5 cm). The apparatus was elevated 75 cm above the floor by a pole fixed underneath the central square. The illumination level was set at approx. 100 lux in the centre of the maze. For testing, each mouse was placed at the end of a closed arm (distal to the centre) facing the wall and was allowed to explore the maze for 5 min. A camera was mounted above the centre of the maze to video-monitor each trial by a trained observer in an adjacent room. The number of entries into each type of arm (placement of all four paws into an arm defining an entry), latency to enter the open arms as well as the time spent in the open and closed arms were recorded by the observer with a hand-held computer. After each trial, the test arena was cleaned carefully with a disinfectant.

The forced swimming procedure was adapted from Ebner (Ebner et al., Eur J Neurosci, 15, 384-388 (2002)). The forced swimming apparatus consisted of a cylindrical 10 L glass tank (24.5 cm in diameter) filled with water (25±1° C.) to a depth of 20 cm. A trained observer recorded the animal's behaviour in moderate lighting conditions (30 lux) for 6 min with a hand-held computer according to one of the following behaviours: (1) struggling, defined as movements during which the forelimbs broke the water's surface; (2) swimming, defined as movement of the animal induced by movements of the fore and hind limbs without breaking the water surface; and (3) floating, defined as the behaviour during which the animal used limb movement just to keep its equilibrium without any movement of the trunk. After each trial, first the mouse was dried with a tissue and put in a new cage, second the water was renewed before continuing with testing.

Motor coordination and balance was assessed using a rotating rod apparatus. The rod diameter was approx. 4.5 cm made of hard plastic material covered by soft black rubber foam with lane widths of 5 cm. The test phase consisted of three trials separated by 15 min intertrial intervals (ITI). Per each trial, three mice were placed on the rod leaving an empty lane between two mice. The rod was initially rotating at constant speed (4 rpm) to allow positioning of all mice in their respective lanes. Once all mice were positioned, the trial was started and the rod accelerated from 4 rpm to 40 rpm in 300 sec. The latency and the speed at which each mouse fell off the rod was measured. Passive rotations were counted as a fall off and the mouse was removed from the rod carefully. After each trail the apparatus was desinfected and dried.

Data were statistically analysed using SPSS software (SPSS Science Software GmbH, Erkrath, Germany). The chosen level of significance was p<0.05.

### EXAMPLE 3

### Phosphorylation of the GABA_{A} Receptor Subunit α2 Through the MAPK Pathway

The MAPK/ERK pathway mainly consists of the three Ser/Thr kinases B-Raf, Mek and Erk which transduce extracellular signals from membrane receptors to nuclear effectors by phosphorylating and thereby activating one after another. By knocking out B-Raf the signal cascade is interrupted, which then leads to a reduced level of activated Erk2 and thereby the activation of downstream targets is blocked. The consensus sequence for the phosphorylation by Erk2 (Pro-Xaa-Ser/Thr-Pro) is already known and can be found in many proteins like c-Fos, p53, STATs, Tau, etc.

Also the amino acids 393/394 of the α2 subunit of the GABA_{A} receptor, which lie in the cytoplasmic loop, show the consensus sequence of an Erk2 phosphorylation site. Additionally, the more upstream amino acids 354-362 comprise the consensus of an Erk2 docking site. Regarding these two facts, the α2 subunit might be a possible target of phosphorylation by the MAPK/ERK pathway. In the B-Raf knockout mouse, the loss of this phosphorylation might be an explanation of the anxiolytic phenotype through a direct or indirect correlation between the GABA_{A} receptor and the MAPK/ERK pathway.

In order to prove the principle of this theory, an in vitro kinase assay was performed. Two 75 aa long oligopeptides were synthesized, one with the correct sequence of amino acids 351-399 of the α2 subunit and another one with the same sequence but with a mutated phosphorylation site (T393V) (FIG. 7).

For the in vitro kinase assay, 10 nmol of each peptide were used with 20 U recombinant p42 MAP Kinase (Erk2) (New England Biolabs), 1×p42 MAP Kinase Reaction Buffer and 10 μCi radioactively labelled γ-³²P-ATP (3000 Ci/mmol, Hartmann Analytics). As controls, reactions without the peptides were used. All reactions were done in triplicate. The assays were incubated for 30 minutes at 30° C. and then stopped by adding Laemmli buffer. The samples were loaded on a 12% Bis-Tris SDS gel and electrophoresed at 200 V for 45 minutes.

Determination of total protein amounts as a loading control was done by Coomassie staining and quantification was done with ImageJ. For measurement of the incorporation of ³²P, the gel was exposed to an imaging plate which was then read out by a Fujifilm FLA-3000 imaging analyzer.

As shown in FIG. 8A, the construct with the wildtype sequence shows a much higher incorporation of ³²P than the second construct with the mutated T393. This demonstrates that the Erk2 kinase has indeed a higher bias for phosphorylating this site in vitro. The background phosphorylation of the mutated construct can be explained by unspecific phosphorylation at all serine/threonine residues due to the excess of Erk2 in the reaction. Comparing the amounts of incorporated ³²P at T393 and at the other Ser/Thr leads to the conclusion that 63% of total phosphorylation occurs at the putative Erk2 site, whereas the other nine Ser/Thr residues are phosphorylated at an average of 4% each (FIG. 8B).

1. A method for identifying a compound capable of modulating an anxiety or depression disorder comprising the steps of:
(a) contacting a composition comprising a B-Raf protein or a B-Raf gene in expressible form or a transcript thereof with a compound under conditions that allow for an interaction of the B-Raf protein or B-Raf gene or a transcript thereof and the compound; and
(b) measuring whether said interaction, if any, results in
i. a change of B-Raf kinase activity compared to B-Raf kinase activity in the absence of said compound;
ii. a modulation of the expression of the B-Raf gene compared to B-Raf gene expression in the absence of said compound; or
iii. the formation of a complex between the compound and the B-Raf protein,
wherein such a change in activity, modulation of expression or the formation of a complex is indicative of the compound being a modulator of an anxiety or depression disorder.

2. The method of claim 1, wherein said composition contains a viable cell comprising said B-Raf protein or said B-Raf gene in an expressible form.

3. The method of claim 1, wherein the change of B-Raf kinase activity is the absence, presence, increase or decrease of said B-Raf kinase activity.

4. The method of claim 1 wherein said modulation of expression results in a higher amount or lower amount of B-Raf protein compared to the amount of B-Raf protein in the absence of said compound.

5. The method of claim 4, wherein the expression of the B-Raf gene is determined by measuring any one of B-Raf transcript level, B-Raf protein level or B-Raf kinase activity.

6. The method of claim 1 comprising a further step
(c) administering the compound suspected to be capable of modulating an anxiety or depression disorder to a non-human mammal and determining whether said compound modulates a B-Raf-mediated process relative to an untreated non-human mammal,
wherein the B-Raf-mediated process is selected from the group consisting of phosphorylation of intracellular or membrane proteins, maintenance of cellular membrane potentials or maintenance of anxiety behaviour and of depression behaviour.

7. The method of claim 1, wherein the modulation of a B-Raf-mediated process results in a decrease of Erk1 and/or Erk2 protein activity.

8. The method of claim 1, wherein the compound is an inhibitor of B-Raf kinase activity or B-Raf gene expression.

9. The method of claim 8, wherein the inhibitor is selected from the group consisting of an antibody, siRNA, shRNA and a small molecule.

10. The method of claim 1, wherein the composition containing a viable cell comprising said B-Raf protein or said B-Raf gene in an expressible form is mounted on a solid support.

11. The method of claim 10, wherein the solid support is a membrane, a glass-, polypropylene- or silicon-chip, are beads or a bead array.

12. The method of claim 2, wherein said cell is part of a tissue.

13. The method of claim 1, wherein the compound can cross the blood-brain barrier.

14. The method of claim 1, wherein the modulation of an anxiety or depression disorder is a reduction of the severity of symptoms or the absence of symptoms associated with said anxiety or depression disorder.

15. A method of treating an anxiety or depression disorder in an individual comprising administering to the individual an effective amount of a compound that inhibits B-Raf kinase activity or inhibits expression of the B-Raf gene.

16. Use of a compound that inhibits B-Raf kinase activity or expression of the B-Raf gene in the manufacture of a pharmaceutical composition for treating an anxiety or depression disorder.

17. The method of claim 15, wherein the compound is selected from the group consisting of Nexavar/BAY 43-9006/Sorafenib, CHIR-265, X-6-(3 acetamidophenyl) pyrazines, 3,5, Di-substituted pyridines, SB-590885 (33), AAL881, LBT613, Omega-carboxypyridyl, Compound 2, ZM 336372, L-779450, PLX4032, 17-allylamino-17-demethoxygeldanarnycin, 17-DMAG, ISIS 5132, LErafAON-ETU, SAHA and NVP-LAQ824.

18. A method of diagnosing a B-Raf-associated anxiety or depression disorder comprising the steps of:
(a) determining the level of B-Raf kinase activity or B-Raf gene expression in a sample obtained from a patient; and
(b) comparing the level of B-Raf kinase activity or B-Raf gene expression obtained in (a) with said levels in a control sample obtained from an individual not affected by a B-Raf-associated anxiety or depression disorder
wherein a change in the level of activity of the B-Raf kinase or of the expression of the B-Raf gene relative to the control sample is indicative of a B-Raf-associated anxiety or depression disorder.

19. The method of claim 18, wherein the sample is selected from the group comprising brain tissue, spinal chord tissue or lymphocytes.

20. The method of claim 18 comprising a further step:
(c) administering an effective amount of a compound that has been identified according to the method of claim 1 to a patient having a B-Raf-associated anxiety or depression disorder.

21. A genetically engineered mouse transgenic for (a) a Cre recombinase gene operatively linked to a CamKIIα promoter and (b) a loxP site flanking each exon boundary of exon 12 of the B-Raf gene obtainable by crossing transgenic line CamKII-CRE-159 with transgenic line B-raf-flox.

22. A method of identifying another gene contributing to the pathophysiology of an anxiety or depression disorder apart from B-Raf comprising the steps of:
(a) crossing the genetically engineered mouse of claim 21 with mice known to harbour mutations in other signaling pathways;
(b) determining the contribution of said signaling pathways in the regulation of anxiety and depression behaviour.

23. The method of any one of claims 1, 18 or 22 wherein the anxiety or depression disorder is selected from the group consisting of generalized anxiety disorder, social phobia, simple phobia, panic disorder, post-traumatic stress disorder (PTSD), obsessive-compulsive disorder (OCD), major depression disorder, dysthymic disorder, bipolar I disorder, bipolar II disorder, cyclothymic disorder, and depressive disorder not otherwise specified.


SEQUENCE LISTING (9 sequences)

SEQ ID NO: 1 - DNA, 2415 bp, mus musculus
atggcggcgctgagtggcggcggtggccgccgcagcggtggcggcggcggcggtggcggcggcggtggcggtggcgacggcggcggcggcgccgagcagggccaggctctgttcaatggcgacatggagccggaggccggcgctggcgccgcggcctcttcggctgcggacccggccattcctgaagaggtatggaatatcaagcaaatgattaagttgacacaggaacatatagaggccctattggacaaatttggtggagagcataacccaccatcaatatacctggaggcctatgaagagtacaccagcaagctagatgcccttcagcaaagagaacagcagcttttggaatccctggtttttcaaactcccacagatgcatcacggaacaaccccaagtcaccacagaaacctatcgttagagtcttcctgcccaacaaacagaggacagtggtacccgcaagatgtggtgttacagttcgagacagtctaaagaaagcactgatgatgagaggtctcatcccagaatgctgtgctgtttacagaattcaggatggagagaagaaaccaattggctgggacacggacatttcctggcttactggagaggagttacatgttgaagtactggagaatgtcccacttacaacacacaactttgtacggaaaacttttttcaccttagcattttgtgacttttgccgaaagctgcttttccagggtttccgttgtcaaacatgtggttataaatttcaccagcgttgtagtacagaggttccactgatgtgtgtaaattatgaccaacttgatttgctgtttgtctccaagttctttgagcatcacccagtaccacaggaggaggcctccttcccagagactgcccttccatctggatcctcttccgcacccccctcagactctactgggccccaaatcctcaccagtccatctccttcaaaatccattccaattccacagcccttccgaccagcagatgaagatcatcgcaatcagtttgggcaacgagaccggtcctcctcagctcccaatgttcatataaacacaattgagcctgtgaatatcgatgaaaaattcccagaagtggaattacaggatcaaagggatttgattagagaccaggggtttcgtggtgatggagcccccttgaaccaactgatgcgctgtcttcggaaataccaatcccggactcccagccccctcctccattctgtccccagtgaaatagtgtttgattttgagcctggcccagtgttcagagggtcaaccacaggcttgtccgccaccccgcctgcctcattacctggctcactcactaacgtgaaagccttacagaaatctccaggtcctcagcgggaaaggaagtcatcttcttcctcatcctcggaggacagaagtcggatgaaaacacttggtagaagagattcaagtgatgactgggagattcctgatggacagattacagtgggacagagaattggatctgggtcatttggaactgtctacaagggaaagtggcatggtgatgtggcagtgaaaatgttgaatgtgacagcacccacacctcaacagctacaggccttcaaaaatgaagtaggagtgctcaggaaaactcgacatgtgaatatcctccttttcatgggctattctacaaagccacaactggcaattgttacacagtggtgtgagggctccagcttatatcaccatctccacatcattgagaccaaatttgagatgatcaaacttatagatattgctcggcagactgcacagggcatggattacttacacgccaagtcaatcatccacagagacctcaagagtaataatatatttcttcatgaagacctcacggtaaaaataggtgactttggtctagccacagtgaaatctcggtggagtgggtcccatcagtttgaacagttgtctggatctattttgtggatggcaccagaagtaatcagaatgcaagataaaaacccgtatagctttcagtcagacgtgtatgcgtttgggattgttctgtacgaactgatgaccggccagctaccttattcaaacatcaacaacagggatcagataatttttatggtgggacgaggatacctatctccagatctcagtaaggtacggagtaactgtccaaaagccatgaagagattaatggcagagtgcctcaaaaagaaaagagacgagagaccactctttccccaaattctcgcctccattgagctgctggcccgctcattgccaaaaattcaccgcagtgcatcagaaccttccttgaatcgggctggtttccaaacagaagattttagtctgtatgcttgtgcttctccgaaaacacccatccaagcagggggatatggagaatttgcagccttcaagtag

SEQ ID NO: 2 - protein, 804 aa, mus musculus
MAALSGGGGRRSGGGGGGGGGGGGGDGGGGAEQGQALFNGDMEPEAGAGAAASSAADPAIPEEVWNIKQMIKLTQEHIEALLDKFGGEHNPPSIYLEAYEEYTSKLDALQQREQQLLESLVFQTPTDASRNNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPVPQEEASFPETALPSGSSSAPPSDSTGPQILTSPSPSKSIPIPQPFRPADEDHRNQFGQRDRSSSAPNVHINTIEPVNIDEKFPEVELQDQRDLIRDQGFRGDGAPLNQLMRCLRKYQSRTPSPLLHSVPSEIVFDFEPGPVFRGSTTGLSATPPASLPGSLTNVKALQKSPGPQRERKSSSSSSSEDRSRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGEFAAFK

SEQ ID NO: 3 - DNA, 2301 bp, homo sapiens
atggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactga

SEQ ID NO: 4 - protein, 766 aa, homo sapiens [sequence omitted]

SEQ ID NO: 5 - DNA, 20 bp, mus musculus
gcatagcgcatatgctcaca

SEQ ID NO: 6 - DNA, 20 bp, mus musculus
ccatgctctaactagtgctg

SEQ ID NO: 7 - DNA, 20 bp, mus musculus
gttgaccttgaactttctcc

SEQ ID NO: 8 - DNA, 23 bp, mus musculus
atgcccaagaagaagaggaaggt

SEQ ID NO: 9 - DNA, 25 bp, mus musculus
gaaatcagtgcgttcgaacgctaga